(19) United States
(12) Patent Application Publication
(10) Pub. No.: US 2016/0346198 A1
(43) Pub. Date: Dec. 1, 2016

(54) NOVEL DISINTEGRATION SYSTEMS FOR PHARMACEUTICAL DOSAGE FORMS

(71) Applicant: Merck Sharp & Dohme Corp., Rahway, NJ (US)

(72) Inventors: Melanie J. Marota, Mount Laurel, NJ (US); Chad David Brown, Quakertown, PA (US); Craig B. Ikeda, Harleysville, PA (US); Mary Ann Johnson, Norristown, PA (US); Hanmi Xi, Furlong, PA (US); Wei Xu, North Wales, PA (US); Lixia Cai, Maple Glen, PA (US)

(73) Assignee: Merck Sharp & Dohme Corp., Rahway, NJ (US)

(21) Appl. No.: 15/116,826

(22) PCT Filed: Feb. 4, 2015
(86) PCT No.: PCT/US15/14405

§ 371 Date: Aug. 4, 2016

Related U.S. Application Data
(60) Provisional application No. 61/936,019, filed on Feb. 5, 2014.
(60) Provisional application No. 62/087,366, filed on Dec. 4, 2014.
(60) Provisional application No. 62/095,427, filed on Dec. 22, 2014.
(60) Provisional application No. 62/095,398, filed on Dec. 22, 2014.

(51) Int. Cl. A61K 9/00; A61K 9/20; A61K 31/4545; A61K 31/5365; A61K 31/551; A61K 9/28; A61K 38/06

(52) U.S. Cl. CPC A61K 9/0053; A61K 9/28; A61K 9/2009; A61K 38/06; A61K 9/2027; A61K 9/2059; A61K 9/2054; A61K 31/5365; A61K 31/551; A61K 31/4545

(57) ABSTRACT

The present disclosure is directed to disintegration systems for solid pharmaceutical dosage forms, which allow rapid disintegration of solid dosage forms that comprise solid dispersion formulations that include pharmaceutically active agents, polymers and optionally surfactants. The present disclosure is also directed to solid pharmaceutical dosage forms, such as tablets, comprising solid dispersion formulations and the disintegration systems, to methods for preparing the disintegration systems, and to methods for preparing the solid pharmaceutical dosage forms.

### CROSS-REFERENCE TO RELATED APPLICATIONS

This application claims the benefit of U.S. Provisional Patent Application No. 61/936,019, filed Feb. 5, 2014; U.S. Provisional Patent Application No. 62/087,366, filed Dec. 4, 2014; U.S. Provisional Patent Application No. 62/095,398, filed Dec. 22, 2014; and U.S. Provisional Patent Application No. 62/095,427, filed Dec. 22, 2014. The disclosures of these applications are incorporated herein in their entirety.

### FIELD OF THE INVENTION

The instant disclosure relates to disintegration systems that are useful for promoting rapid disintegration of pharmaceutical dosage forms, particularly those comprising poorly soluble pharmaceutical active ingredients that are formulated in solid dispersions or solid solutions. In particular, the disintegration systems are two component systems that comprise a disintegrant and a salt, where the salt is in a particulate form.

### BACKGROUND OF THE INVENTION

The development of oral dosage forms of active pharmaceutical ingredients (APIs, drugs or drug substances) can be a challenge. The disintegration and dissolution properties of the oral dosage forms can have profound effects on the efficacy of drug substances. See Filippos Kesisoglou et al., Development of In Vitro-In Vivo Correlation for Amorphous Solid Dispersion Immediate-Release Suvorexant Tablets and Application to Clinically Relevant Dissolution Specifications and In-Process Controls, J. PHARM SCI. 1 (2015). When the drug is poorly water soluble, as in the case of so-called Class II and Class IV drugs under the Biopharmaceutics Classification System (see e.g. The Biopharmaceutics Classification System (BCS) Guidance, http://www.fda.gov/AboutFDA/CentersOffices/OfficeofMedicalProducts andTobacco/CDER/ucm128219.htm; B. Basanta Kumar Reddy and A. Karunakar, Biopharmaceutics Classification System: A Regulatory Approach, DISSOLUTION TECHNOLOGIES 31-37 (February 2011)), the effects of disintegration on the efficacy of the drug is more profound. See Filippos Kesisoglou et al., Development of In Vitro-In Vivo Correlation for Amorphous Solid Dispersion Immediate-Release Suvorexant Tablets and Application to Clinically Relevant Dissolution Specifications and In-Process Controls, J. PHARM SCI. 1 (2015).

Oral dosage forms containing solid solutions or solid dispersions, which comprise a drug substance and optionally a surfactant dispersed in a polymer matrix, have been used to promote the oral absorption of poorly water soluble APIs. See James Ford, The Current State of Solid Dispersions, 61(1) PHARM. ACTA HELV. 69-88 (1986). Solid solutions and solid dispersions (in which the API forms a homogeneous or nearly homogeneous glass in the excipient matrix) are believed to improve the absorption of orally administered API by improving: (i) the wetting properties of the API; (ii) causing at the point of absorption transient supersaturation of the API with respect to a lower energy (e.g. crystalline) phase API; or (iii) both effects. In general, solid solutions and solid dispersions are believed to enable drug absorption by enhancing the dissolution rate and/or the extent to which the drug is dissolved from the matrix. See generally Ladan Akbarpour Nikghalb et al., Solid Dispersion: Methods and Polymers to Increase the Solubility of Poorly Soluble Drugs, 2(10) J. APP. PHARM. SCI. 170-175 (2012).

In order for these solid solutions or solid dispersions to be effective, the oral dosage form in which they are contained must disintegrate at an appropriate point in the digestive system, in the stomach or intestines, and release sufficient amounts of the drug substance to provide absorption and efficacy. The use of disintegrants to promote the breakup of oral dosage forms such as tablets or capsules into smaller fragments is well known. The disintegrants act by swelling, wicking deformation and/or other disruptive forces to breakup tablets and capsule contents into granules. See P. S. Mohanachandran et al., Superdisintegrants: An Overview, 6(1) INTL J. PHARM. SCI. REV. & RES. 105-109 (2011).

Improved disintegrants and disintegration systems in which a disintegrant and a salt are employed for use in pharmaceutical dosage forms have been the subject of research. See P.S. Mohanachandran et al., Superdisintegrants: An Overview, 6(1) INTL J. PHARM. SCI. REV. & RES. 105-109 (2011); Atsushi Kajiyama et al., Improvement of HPMC Tablet Disintegration by Addition of Inorganic Salts, 56(4) CHEM. PHARM. BULL. 598-601; Justin R. Hughey et al., The Use of Inorganic Salts to Improve the Dissolution Characteristics of Tablets containing SOLPLUS®-based Solid Dispersions, 48 EUROPEAN J. PHARM. SCI. 758-766 (2013); U.S. Patent Application Publication No. US2008/0214557A1; U.S. Patent Application Publication No. US2002/0031547; U.S. Pat. No. 7,189,415.

Superdisintegrants have been described in the literature as an additive to tablet formulations to increase the disintegration rate of tablets and include modified starches, cross-linked polyvinylpyrrolidones, modified celluloses, soy polysaccharides, cross-linked alginic acids, gellan gums, xanthan gums, and calcium silicates. See P. S. Mohanachandran et al., Superdisintegrants: An Overview, 6(1) INTL J. PHARM. SCI. REV. & RES. 105-109 (2011). These disintegrants alone do not always allow for optimum disintegration properties of tablets at levels that allow for sufficient mechanical integrity. As described in U.S. Patent Application Publication No. US2008/0214557A1, it has been demonstrated that the addition of a salt to a formulation can increase the effectiveness of disintegrants in tablet formulations containing crystalline active ingredients that are electrically neutral or positively charged, acidic or an acidic salt of a basic compound. Similarly, the use of effervescent salts with or without disintegrants to improve the disintegration of tablets containing solid dispersions of low soluble compounds comprised of gel forming cellulosic-derivative polymer, specifically HPMC, in which the effervescence aids in the disruption of the gel network, has been described. See U.S. Patent Application Publication No. US2002/0031547; U.S. Pat. No. 7,189,415; Atsushi Kajiyama et al., Improvement of HPMC Tablet Disintegration by Addition of Inorganic Salts, 56(4) CHEM. PHARM. BULL. 598-601. Additionally, the inclusion of inorganic salts, preferably effervescent salts like potassium bicarbonate, to improve the disintegration of tablets containing solid dispersions comprised of SOLUPLUS® have been studied. See Justin R. Hughey et al., The Use of Inorganic Salts to Improve the Dissolution Characteristics of Tablets containing SOLUPLUS®-based Solid Dispersions, 48 EUROPEAN J. PHARM. SCI. 758-766 (2013). It was found the inclusion of kosmotropic salts allowed for the rapid hydration of the entire tablet and the formation of a gel structure with gel strength dependent on the type of salt utilized; potassium bicarbonate provided the advantage of a weak gel network while generating carbon dioxide bubbles in acidic medium to aid in the disruption of the gel and providing fast disintegration. See Justin R. Hughey et al., The Use of Inorganic Salts to Improve the Dissolution Characteristics of Tablets containing SOLUPLUS®-based Solid Dispersions, 48 EUROPEAN J. PHARM. SCI. 758-766 (2013).

The use of solid dispersion formulations to effectively promote oral drug absorption continues to grow, but their design remains largely a matter of trial and error. There remains a need for oral dosage forms that employ solid dispersion formulations of drug substances and that may provide effective absorption following oral administration, which is useful to reduce pill burden (e.g., the number of tablets administered) and regimen complexity (e.g., eliminating the need to administer with food), and to facilitate co-dosing with other medications, such as antacid medications. Formulations with this type of enhanced absorption will ultimately improve compliance and, therefore, efficacy.

However, formulating poorly soluble drugs into oral dosage forms that disintegrate rapidly continues to be a challenge for formulators. Many APIs are not chemically stable in the presence of basic effervescent salts, such as described above. In addition, large oral dosage forms, which may be difficult for patients to swallow, may have negative implications for safety and patient compliance. See e.g. Guidance for Industry: Size, Shape, and Other Physical Attributes of Generic Tablets and Capsules (Draft Guidance, U.S. Department of Health and Human Services, Food and Drug Administration, Center for Drug Evaluation and Research (CDER), December 2013), http://www.fda.gov/downloads/drugs/guidance/complianceregulatory information/guidances/ucm377938.pdf. Incorporation of solid dispersions and solid solutions into smaller dosage forms while preserving rapid disintegration rates can be particularly challenging because higher loading of the solid dispersion or solid solution—necessary to increase drug loading—inherently increases the concentration of solid dispersion polymer in the dosage form. Thus, there remains a need for improved disintegration systems for pharmaceutical oral dosage forms containing solid solution formulations and for improved oral dosage forms that can maximize the advantages of solid solution formulations by rapid disintegration.

The current invention relates to novel disintegration systems that comprise disintegrants and particulate inorganic salts and that may provide improved disintegration of oral dosage forms that include solid dispersion formulations, and to novel oral dosage forms that comprise solid dispersion formulations and the novel disintegration systems, that may provide improved oral absorption and enhanced dissolution rates.

### SUMMARY OF THE INVENTION

The present disclosure relates to disintegration systems for pharmaceutical formulations comprising a) a disintegrant selected from the group consisting of modified starches, cross-linked polyvinylpyrrolidones, modified celluloses, soy polysaccharides, cross-linked alginic acids, gellan gum, xantham gum, calcium silicate and ion exchange resins; and b) an inorganic salt, where the inorganic salt is in the form of particles, wherein said particles are characterized by (i) a d₅₀ value of less than about 325 micron; (ii) a d₁₀ value of less than about 185 micron; and (iii) a d₉₀ value of less than about 460 micron; wherein the disintegrant and the inorganic salt are provided in a ratio of from about 2:1 to about 1:3. Such disintegration systems may allow salt dissolution at a rate that is high enough to provide high local ionic strength, which may disrupt gel layer formation without the need for effervescence. Embodiments, including embodiments of pharmaceutical dosage forms that comprise such disintegration systems, may provide improved oral bioavailability for pharmaceutically active agents.

Other embodiments, aspects and features of the present invention are either further described in or will be apparent from the ensuing description, examples and appended claims.

### BRIEF DESCRIPTION OF THE DRAWINGS

FIG. 1 provides a schematic representation of the formulation process for preparing the solid dispersion intermediate of Compound A as set forth in Example 2.

FIG. 2 provides a schematic representation of the formulation process for preparing Formulation 2a of Example 2.

FIG. 3 provides a graphical representation of comparative release rates of croscarmellose sodium-containing formulations according to Example 10.

FIG. 4 provides a graphical representation of comparative release rates of copovidone-containing formulations according to Example 10.

FIG. 5 provides a graphical representation of comparative release rates of formulations according to Example 11.

### DETAILED DESCRIPTION OF THE INVENTION

The present disclosure relates to disintegration systems for pharmaceutical formulations comprising a) a disintegrant selected from the group consisting of modified starches, cross-linked polyvinylpyrrolidones, modified celluloses, soy polysaccharides, cross-linked alginic acids, gellan gum, xantham gum, calcium silicate and ion exchange resins; and b) an inorganic salt, where the inorganic salt is in the form of particles, wherein said particles are characterized by (i) a d₅₀ value of less than about 325 micron; (ii) a d₁₀ value of less than about 185 micron; and (iii) a d₉₀ value of less than about 460 micron; wherein the disintegrant and the inorganic salt are provided in a ratio of from about 2:1 to about 1:3. The disclosure is also directed to granulation intermediates comprising such disintegration systems, to blended compositions comprising such disintegration systems and to oral dosage forms, such as tablets or capsules, comprising the disintegration systems.

In the embodiments of the dispersion systems, granulation intermediates, blended compositions, and oral dosage forms provided above, it is to be understood that each embodiment may be combined with one or more other embodiments, to the extent that such a combination provides a stable formulation and is consistent with the description of the embodiments. It is further to be understood that the embodiments of compositions and methods provided above are understood to include all embodiments of the formulations, including such embodiments as result from combinations of embodiments.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents.

As used herein, the term “one or more” item includes a single item selected from the list as well as mixtures of two or more items selected from the list.

As used herein, the term “amorphous” indicates that the material lacks order on a molecular level and may exhibit the physical properties of a solid or a liquid, depending on the temperature of the material. Amorphous materials do not give distinctive X-ray diffraction patterns.

As used herein, the term “crystalline” indicates that the material has a regular ordered internal structure at the molecular level when in the solid phase, and the crystalline material gives a distinctive X-ray diffraction pattern with defined peaks.

As used herein, the term “substantially amorphous” refers to a composition in which greater than 70%; or greater than 75%; or greater than 80%; or greater than 85%; or greater than 90%; or greater than 95%, or greater than 99% of the compound is amorphous. “Substantially amorphous” can also refer to material that has no more than about 20% crystallinity, or no more than about 10% crystallinity, or no more than about 5% crystallinity, or no more than about 2% crystallinity.

The term “effective amount” indicates a sufficient amount to exert a therapeutic or prophylactic effect.

The term “formulation”, as used herein, refers to a blend, aggregation, solution or other combination of materials which includes an active pharmaceutical ingredient (API) which formulation is adapted to a particular mode of administration, for example, a formulation suitable for pressing into tablets designed for oral administration, in the treatment, management, prevention and etc. of a disease state or condition in a patient.

Pharmaceutical formulations intended for the preparation of oral dosage forms (tablets and capsules) may further contain one or more agents selected from the group consisting of sweetening agents, flavoring agents, coloring agents and preserving agents in order to provide pharmaceutically elegant and palatable preparations.

The term “subject” (alternatively referred to herein as “patient”) as used herein refers to an animal, preferably a mammal, most preferably a human, who has been the object of treatment, observation or experiment. When a human subject suffering from the condition to be treated is included in the activity they are alternatively referred to herein as a “patient”.

The term “pharmaceutically acceptable salt” refers to a salt of the parent compound that has activity and that is not biologically or otherwise undesirable (e.g., is neither toxic nor otherwise deleterious to the recipient thereof); also included in this term are complexes that comprise solvent molecules and a salt of the parent compound. Suitable salts include acid addition salts that may, for example, be formed by mixing a solution of a compound with a solution of a pharmaceutically acceptable acid such as hydrochloric acid, sulfuric acid, acetic acid, trifluoroacetic acid, benzoic acid, phosphoric acid, methanesulfonic acid, naphthalene-1,5-disulfonic acid or toluenesulfonic acid. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands, such as quaternary ammonium salts. Also, in the case of an acid (—COOH) or alcohol group being present, pharmaceutically acceptable esters can be employed to modify the solubility or hydrolysis characteristics of the compound.

The term “polymer” as used herein refers to a chemical compound or mixture of compounds consisting of repeating structural units created through a process of polymerization.

Suitable polymers useful in this invention are described throughout. When specific polymers that are suitable for use in the compositions of the present invention are blended, the blends of such polymers may also be suitable. Thus, the term “polymer” is intended to include blends of polymers in addition to a single species of polymer.

### Disintegration Systems

Embodiments of the disclosure provide disintegration systems for pharmaceutical formulations comprising a) a disintegrant selected from the group consisting of modified starches, cross-linked polyvinylpyrrolidones, modified celluloses, soy polysaccharides, cross-linked alginic acids, gellan gum, xantham gum, calcium silicate and ion exchange resins; and b) an inorganic salt, where the inorganic salt is in the form of particles, wherein said particles are characterized by (i) a d₅₀ value of less than about 325 micron; (ii) a d₁₀ value of less than about 185 micron; and (iii) a d₉₀ value of less than about 460 micron; wherein the disintegrant and the inorganic salt are provided in a ratio of from about 2:1 to about 1:3. A “disintegration system” is a combination of at least one disintegrant and at least one inorganic salt, the combination of which provides beneficial anti-gellation effects. Disintegration systems such as those described and claimed herein may allow salt dissolution at a rate that is high enough to provide high local ionic strength, which may disrupt gel layer formation without the need for effervescence. In embodiments, the at least one disintegrant and at least one salt may be mixed to provide a single additive form. In alternative embodiments, the at least one disintegrant and at least one salt are provided as individual components into the desired composition or formulation.

A “disintegrant” is an excipient that expands and/or dissolves when placed in an aqueous environment, for example, the gastrointestinal tract, which aids a tablet in breaking apart and promotes release of an active pharmaceutical ingredient contained in a tablet. In embodiments of this disclosure, the disintegrant that is provided in the disintegration system may be selected from so-called “superdisintegrants”. See P. S. Mohanachandran et al., Superdisintegrants: An Overview, 6(1) INTL J. PHARM. SCI. REV. & RES. 105-109 (2011).

Examples of superdisintegrants include but are not limited to modified starches, such as sodium carboxylmethyl starch and cross-linked starches, such as commercially available starches including EXPLOTAB® and PRIMOGEL®; cross-linked polyvinylpyrrolidones, such as crospovidone and commercially available cross-linked polyvinylpyrrolidones including CROSSPOVIDONE M®, KOLLIDON®, and POLYPLASDON®; modified celluloses, such as croscarmellose sodium and commercially available cross-liked celluloses including CROSSCARMELLOSE AC-DI-SOL®, NYMCE ZSX®, PRIMELLOSE®, SOLUTAB®, VIVASOL®, and L-HPC; soy polysaccharides, such as commercially available EMCOSOY®; cross-linked alginic acids, such as commercially available ALGINIC ACID NF; gellan gum; xantham gum; calcium silicate; and ion exchange resins. In particular embodiments, the disintegrant is selected from the group consisting of sodium carboxylmethyl starch, crospovidone, croscarmellose sodium, calcium silicate and ion exchange resins. In even more particular embodiments, the disintegrant is selected from crospovidone and croscarmellose sodium.

The inorganic salt that is provided in the disintegration system is in a particulate form, and in embodiments, the inorganic salt may be in powder form. In particular embodiments, the inorganic salt may be in the form of a powder, wherein the powder is characterized by (i) a d₅₀ value of less than about 210 micron; (ii) a d₁₀ value of less than about 50 micron; and (iii) a d₉₀ value of less than about 470 micron. The powder form may provide additional benefits in some embodiments; the increased surface to volume ratio of the powder form may provide more rapid salt dissolution and thus may provide more rapid increases in local ionic strength that in turn may provide improved gel disruption.

In embodiments, the inorganic salt that is provided in the claimed disintegration systems may be selected from sodium chloride (NaCl), potassium chloride (KCl), potassium carbonate (K₂CO₃), sodium carbonate (Na₂CO₃), sodium bicarbonate (NaHCO₃), sodium sulfate (Na₂SO₄), calcium chloride (CaCl₂), potassium phosphate (KH₂PO₄), sodium phosphate (dibasic) (NaH₂PO₄), and potassium sulfate (K₂SO₄), and combinations thereof. In particular embodiments, the inorganic salt is a single salt selected from the group consisting of NaCl, KCl, K₂CO₃, Na₂CO₃, NaHCO₃, Na₂SO₄, CaCl₂, KH₂PO₄, NaH₂PO₄, and K₂SO₄. In still further specific instances of this second aspect, the inorganic salt is selected from the group consisting of NaCl and KCl.

In combination, the disintegrant and the particulate inorganic salt of the claimed disintegration systems demonstrate an unexpected synergy that provides rapid disintegration of the pharmaceutical dosage forms in which they are employed. As described and demonstrated in the examples, the combination of disintegrant and particulate inorganic salt allowed for faster disintegration of tablets containing solid dispersions over tablets with either component alone. This is of particular interest for indications that require a rapid onset of action (pain, insomnia, etc.) or have a limited absorption window (for example, where absorption is isolated to the upper gastrointestinal tract).

Surprisingly, the increased disintegration rate of tablets containing the combination of disintegrant and particulate inorganic salt allowed tablets with higher dispersion loadings to be realized while maintaining disintegration times appropriate for immediate release dosage forms. This is of particular interest for indications or patient markets requiring smaller tablet images, higher doses, and/or fixed-dose combinations with other active ingredients.

Further, the combination of disintegrant and salt allowed combinations of dispersions to be co-processed within the same tablet while maintaining disintegration times appropriate for immediate release dosage forms. This is of particular interest for indications requiring fixed-dose combinations of multiple poorly soluble APIs (for example, in treatment of hepatitis C virus and human immunodeficiency virus, etc.).

### Solid Dispersion Formulations

The disintegration systems described above are useful in combination with solid dispersion formulations, which comprise (a) one or more API or a pharmaceutically acceptable salt thereof; (b) one or more pharmaceutically acceptable polymers; and (c) optionally one or more pharmaceutically acceptable surfactants. In embodiments, one solid dispersion formulation may be included in the blended compositions or oral dosage forms described. In additional embodiments, two or more different solid dispersion formulations, each with independently selected API, polymer and optional surfactant (if present) may be included in the blended compositions or oral dosage forms described.

The relative amount of drug, polymer and optional surfactant can vary widely. The optimal amount of the polymer and optional surfactant can depend, for example, on the hydrophilic lipophilic balance (HLB), melting point, and water solubility of the copolymer, and the surface tension of aqueous solutions of the surfactant, etc.

### Drug Substance(s)

In particular, the drug substance or API or pharmaceutically acceptable salt thereof is poorly water soluble. Exemplary drug substances that may be included in the solid dispersion formulation(s) include, but are not limited to, antiviral compounds, compounds that are useful for pain management and compounds for sleep regulation in mammals. These exemplary drug substances include, but are not limited to hepatitis C virus (HCV) protease inhibitors, HCV polymerase inhibitors, HCV NS4A inhibitors, HCV NS5A inhibitors, HCV NS5b inhibitors, human immunodeficiency virus (HIV) inhibitors, calcitonin gene-related peptide (CGRP) antagonists, and orexin receptor antagonists.

HCV protease inhibitors include, but are not limited to, those disclosed in U.S. Pat. Nos. 8,080,654; 7,973,040; 8,828,930; 8,927,569; 7,879,797; 7,470,664; 8,216,999; 8,377,873; 8,278,322; 8,138,164; 8,377,874; 8,309,540; 8,591,878; 7,494,988; 7,485,625; 7,795,250; 7,449,447; 7,442,695; 7,425,576; 7,342,041; 7,253,160; 7,244,721; 7,205,330; 7,192,957; 7,186,747; 7,173,057; 7,169,760; 7,012,066; 6,914,122; 6,911,428; 6,894,072; 6,846,802; 6,838,475; 6,800,434; 6,767,991; 5,017,380; 4,933,443; 4,812,561 and 4,634,697; U.S. Patent Application Publication Nos. US2014/0057836, US2013/0178413, US2010/0099695, US2014/0296136, US2002/0068702, US2002/0160962, US2005/0119168, US2005/0176648, US2005/0209164, US2005/0249702 and US2007/0042968; and PCT International Patent Application Publication Nos. WO2014/025736, WO2009/010804, WO2010/011566, WO02011/014487, WO2006/119061, WO2007/015855, WO2007/015787, WO2007/016441, WO2007/131966, WO2007/148135, WO2008/057209, WO2008/051475, WO2008/057208, WO2008/051514, WO2009/108507, WO2008/051477, WO2012/040040, WO02013/074386, WO3/006490, WO03/087092, WO04/092161 and WO08/124148. HCV protease inhibitors also include, but are not limited to, grazoprevir, vaniprevir, boceprevir, narlaprevir (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix). Further examples of HCV protease inhibitors include, but are not limited to, those disclosed in James A. Landro et al., Mechanistic Role of an NS4A Peptide Cofactor with the Truncated NS3 Protease of Hepatitis C Virus: Elucidation of the NS4A Stimulatory Effect via Kinetic Analysis and Inhibitor Mapping, 36(31) BIOCHEMISTRY 9340-9348 (1997); Paolo Ingallinella et al., Potent Peptide Inhibitors of Human Hepatitis C Virus NS3 Protease Are Obtained by Optimizing the Cleavage Products, 37(25) BIOCHEMISTRY 8906-8914 (1998); Montse Llinàs-Brunet et al., Peptide-Based Inhibitors of the Hepatitis C Virus Serine Protease, 8(13) BIOORG. MED. CHEM. LETT. 1713-1718 (1998); Franck Martin et al., Design of Selective Eglin Inhibitors of HCV NS3 Proteinase, 37(33) BIOCHEMISTRY 11459-11468 (1998); Nazzareno Dimasi et al., Characterization of Engineered Hepatitis C Virus NS3 Protease Inhibitors Affinity Selected from Human Pancreatic Secretory Trypsin Inhibitor and Minibody Repertoires, 71(10) J. VIROL. 7461-7469 (1997); Martin et al., Affinity Selection of a Camelized VH Domain Antibody Inhibitor of Hepatitis C Virus NS3 Protease, 10(5) PROTEIN ENG. 607-614 (1997); Abdul-Nasser Elzouki et al., Serine Protease Inhibitors in Patients with Chronic Viral Hepatitis, 27(1) J. HEPAT. 42-48 (1997); U.S. Patent Application Publication Nos. US2005/0249702 and US 2007/0274951; and PCT International Patent Application Publication Nos. WO98/14181, WO98/17679, WO98/17679, WO98/22496, WO99/07734 and WO05/087731.

HCV polymerase inhibitors include, but are not limited to, VP-19744 (Wyeth/ViroPharma), PSI-7851 (Pharmasset), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716 (ViroChem), GSK-71185 (Glaxo SmithKline), ANA598 (Anadys), GSK-625433 (Glaxo SmithKline), XTL-2125 (XTL Biopharmaceuticals), and those disclosed in Zhi-Jie Ni et al., Progress and development of small molecule HCV antivirals, 7(4) CURRENT OPINION IN DRUG DISCOVERY AND DEVELOPMENT 446 (2004); Seng-Lai Tan et al., Hepatitis C therapeutics: current status and emerging strategies, 1 NATURE REVIEWS 867 (2002); and Pierre L. Beaulieu et al., Inhibitors of the HCV NS5B Polymerase: New Hope for the Treatment of Hepatitis C Infections, 5 CURRENT OPINION IN INVESTIGATIONAL DRUGS 838 (2004).

HCV NS4A inhibitors include, but are not limited to, those disclosed in U.S. Pat. Nos. 7,476,686 and 7,273,885; U.S. Patent Application Publication No. US2009/0022688; and PCT International Patent Application Publication Nos. WO2006/019831 and WO2006/019832. Additional HCV NS4A inhibitors include, but are not limited to, AZD2836 (Astra Zeneca) and ACH-806 (Achillon Pharmaceuticals, New Haven, Conn.).

HCV NS5A inhibitors include, but are not limited to, those disclosed in U.S. Pat. Nos. 8,871,759 and 8,609,635; U.S. Patent Application Publication No. US2014/0371138; and PCT International Patent Application Publication Nos. WO2014/110705 and WO2014/110706.

HCV NS5B inhibitors include, but are not limited to, those disclosed in U.S. Patent Application Publication No. US2012/0328569; and PCT International Patent Application Publication Nos. WO2010/111483, WO2011/106992, WO2011/106985, WO2011/106929, and WO2013/033971.

Calcitonin Gene-Related Peptide antagonist compounds, which are directed at pain indications, for example, management, prevention, or alleviation of migraine conditions, include, for example, but are not limited to, compounds described in PCT International Patent Application Publication Nos. WO2013/036861; WO2008/112159; WO2008/130524; WO2014/195848; WO2004/082605; WO2004/082678; WO2004/083187; WO2004/082602; WO2006/031513; WO2006/031606; WO2006/031610; WO2006/031491; WO2006/029153; WO2006/031676; WO2007/061676; WO2007/061694; WO2007/061695; WO2007/061677; WO2007/061692; WO2007/061696. CGRP antagonist compounds include, but are not limited to, those described in U.S. Pat. Nos. 8,481,556; 8,754,096; and 8,912,210. In principle, any CGRP compound that is a Class II or Class IV compound is suitable for inclusion in the present invention.

Orexin receptors are found in the mammalian brain and the scientific literature suggests that they may be involved in various pathologies such as depression; anxiety; addictions; obsessive compulsive disorder; affective neurosis; depressive neurosis; anxiety neurosis; dysthymic disorder; behavior disorder; mood disorder; sexual dysfunction; psychosexual dysfunction; sex disorder; schizophrenia; manic depression; delirium; dementia; severe mental retardation and dyskinesias such as Huntington's disease and Tourette syndrome; eating disorders such as anorexia, bulimia, cachexia, and obesity; addictive feeding behaviors; binge/purge feeding behaviors; cardiovascular diseases; diabetes; appetite/taste disorders; emesis, vomiting, nausea; asthma; cancer; Parkinson's disease; Cushing's syndrome/disease; basophile adenoma; prolactinoma; hyperprolactinemia; hypophysis tumor/adenoma; hypothalamic diseases; inflammatory bowel disease; gastric diskinesia; gastric ulcers; Froehlich's syndrome; adrenohypophysis disease; hypophysis disease; adrenohypophysis hypofunction; adrenohypophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; disturbed biological and circadian rhythms; sleep disturbances associated with diseases such as neurological disorders, neuropathic pain and restless leg syndrome; heart and lung diseases, acute and congestive heart failure; hypotension; hypertension; urinary retention; osteoporosis; angina pectoris; myocardinal infarction; ischemic or haemorrhagic stroke; subarachnoid haemorrhage; ulcers; allergies; benign prostatic hypertrophy; chronic renal failure; renal disease; impaired glucose tolerance; migraine; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; emesis, nausea, vomiting; conditions associated with visceral pain such as irritable bowel syndrome, and angina; migraine; urinary bladder incontinence e.g. urge incontinence; tolerance to narcotics or withdrawal from narcotics; sleep disorders; sleep apnea; narcolepsy; insomnia; parasomnia; jet lag syndrome; and neurodegenerative disorders including nosological entities such as disinhibition-dementia-parkinsonism-amyotrophy complex; pallido-ponto-nigral degeneration; epilepsy; seizure disorders and other diseases related to general orexin system dysfunction. Orexin receptor antagonists compounds that could be included in solid dispersions that are incorporated into blended compositions and oral dosage forms described herein, include, but are not limited to, those disclosed in U.S. Pat. Nos. 7,951,797 and 8,242,121; U.S. Patent Application Publication No. US2008/0132490; PCT International Patent Application Publication Nos. WO2008/069997 and WO2008/147518; Christopher D. Cox et al., Discovery of the Dual Orexin Receptor Antagonist [(7R)-4-(5-Chloro-1,3-benzoxazol-2-yl)-7-methyl-1,4-diazepan-1-yl][5-methyl-2-(2H-1,2,3-triazol-2-yl)phenyl]methanone (MK-4305) for the Treatment of Insomnia, 53(14) J. MED. CHEM. 5320-5332 (2010); Neil A. Strotman et al., Reaction Development and Mechanistic Study of a Ruthenium Catalyzed Intramolecular Asymmetric Reductive Amination en Route to the Dual Orexin Inhibitor Suvorexant (MK-4305), 133(21) J. AM. CHEM. SOC. 836208371 (2011); Carl A. Baxter et al., The First Large-Scale Synthesis of MK-4305: A Dual Orexin Receptor Antagonist for the Treatment of Sleep Disorder, 15(2) ORG. PROCESS RES. & DEV. 367-375 (2011).

In particular embodiments, the one or more drug substance may be independently selected from the following compounds:

(1aR,5S,8S,10R,22aR)-N-[(1R,2S)-1-[(cyclopropylsulfonamido)carbonyl]-2-ethenylcyclopropyl]-14-methoxy-5-(2-methylpropan-2-yl)-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide hydrate (grazoprevir), which is shown below as Compound A:

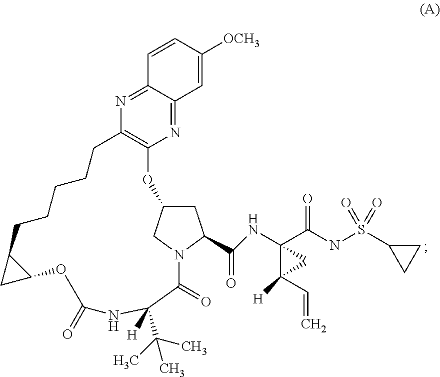 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1]

(4R,7S,10S)-10-tert-Butyl-N-{(1R,2R)-1-[N -(cyclopropanesulfonyl)carbamoyl]-2-ethylcyclopropyl}-15,15-dimethyl-3,9,12-trioxo -6,7,9,10,11,12,14,15,16,17,18,19-dodecahydro-1H,3H,5H -2,23:5,8-dimethano-4,13,2,8,11-benzodioxatriazacyclohenicosine-7-carboxamide (vaniprevir), which is shown as Compound B:

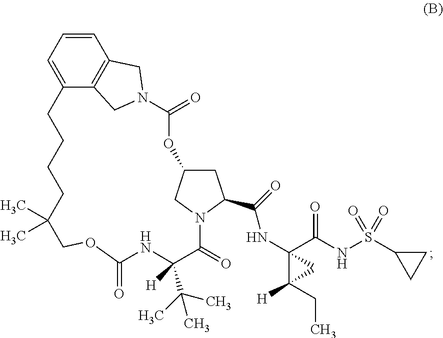

(1R,5S)-N-[3-Amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide (boceprevir), which is shown as Compound C:

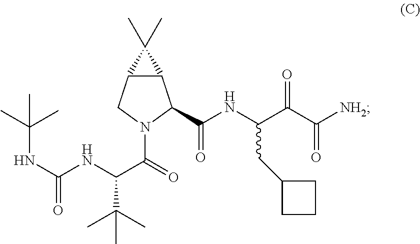

(1R,2S,5S)-3-((S)-2-(3-(1-((tert-butylsulfonyl)methyl)cyclohexyl)ureido) -3,3-dimethylbutanoyl)-N-((S)-1-(cyclopropylamino)-1,2-dioxoheptan-3-yl)-6,6-dimethyl-3-azabicyclo[3.1.0]hexane-2-carboxamide (narlaprevir), which is shown as Compound D:

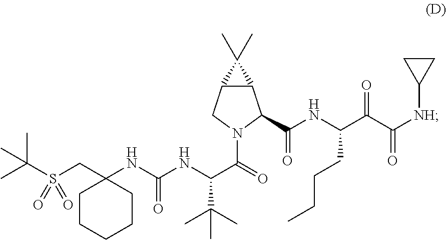

dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate (elbasvir), which is shown below as Compound E:

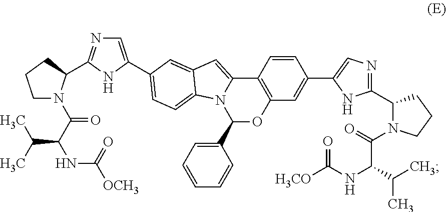 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]

dimethyl ((2S,2′S)-((2S,2′S)-2,2′-(5,5′-(S)-6-(2-cyclopropylthiazol-5-yl) -1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, which is shown as Compound F:

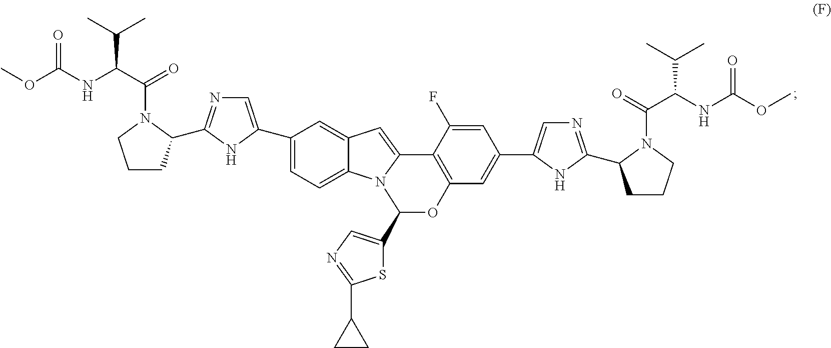

(2R)-isopropyl 2-(((((2R,3R,4R,5R)-4-chloro-5-(2,4-dioxo-3,4-dihydropyrimidin-1(2H)-yl)-3-hydroxy-4-methyltetrahydrofuran-2-yl)methoxy)(phenoxy)phosphoryl)amino)propanoate, which is shown as Compound G:

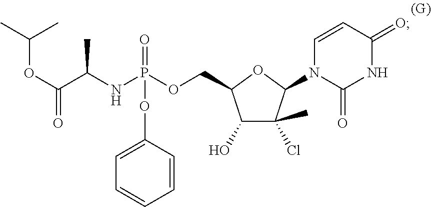 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]

(2R,3 S,4R,5R)-4-cyano-5-(2,4-dioxo-3,4-dihydropyrimidin-1 (2H)-yl)-2-((((((S)-1-isopropoxy-1-oxopropan-2-yl)amino)(phenoxy)phosphoryl)oxy)methyl)-4-methyltetrahydrofuran-3-yl isobutyrate, which is shown as Compound H:

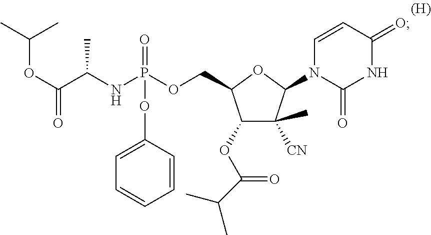 [SMILES: CC(C)OC(=O)[C@H](C)NP(=O)(OC[C@H]1O[C@@H](n2ccc(=O)[nH]c2=O)[C@](C)(C#N)[C@@H]1OC(=O)C(C)C)Oc1ccccc1]

5-(11-fluoro-6H-pyrido[2′,3′:5,6][1,3]oxazino[3,4-a]indol-2-yl)-2-(4-fluorophenyl)-N-methyl-6-(N-methylmethylsulfonamido)benzofuran-3-carboxamide, which is shown as Compound I:

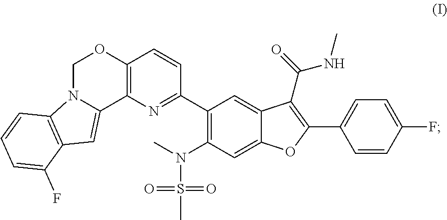 [SMILES: CNC(=O)c1c(-c2ccc(F)cc2)oc2cc(N(C)S(C)(=O)=O)c(-c3ccc4c(n3)-c3cc5c(F)cccc5n3CO4)cc12]

CGRP antagonist compounds that are based on the structure of Formula J:

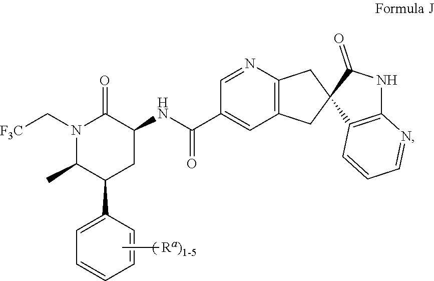

where “R^{a}” is various substituents (for example, Compound J1, where “R^{a}” is hydrogen, (S)-N -((3S,5S,6R)-6-methyl-2-oxo-5-phenyl-1-(2,2,2-trifluoroethyl)piperidin-3-yl)-2′-oxo-1′,2′,5,7-tetrahydrospiro[cyclopenta[b]pyridine-6,3′-pyrrolo[2,3-b]pyridine]-3-carboxamide:

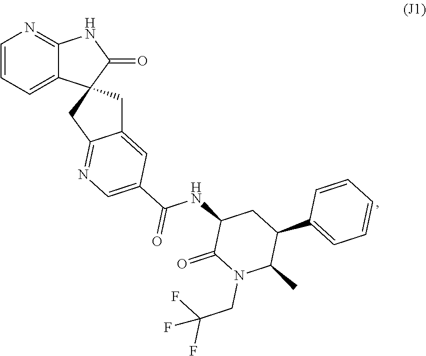 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

and, for example, Compound J2, where three of “R^{a}” are selected to be fluorine, ((S)-N -((3S,5S,6R)-6-methyl-2-oxo-1-(2,2,2-trifluoroethyl)-5-(2,3,6-trifluorophenyl)piperidin-3-yl)-2′-oxo-1′,2′,5,7-tetrahydrospiro[cyclopenta[b]pyridine-6,3′-pyrrolo[2,3-b]pyridine]-3-carboxamide):

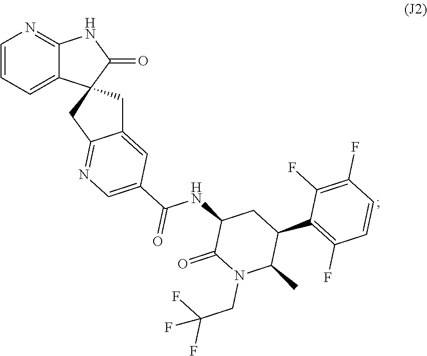 [SMILES: C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

5-chloro-2-{(5R)-5-methyl-4[5-methyl-2-(2H-1,2,3-triazol-2-yl)benzoyl]-1,4-diazepan-1-yl}-1,3-benzoxazole (suvorexant), which is shown as Compound L:

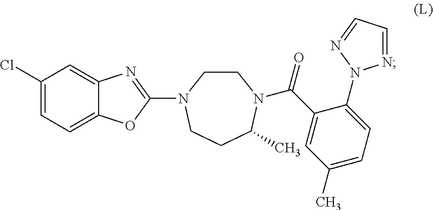 [SMILES: Cc1ccc(-n2nccn2)c(C(=O)N2CCN(c3nc4cc(Cl)ccc4o3)CC[C@H]2C)c1]

and

2-{2-[((2R,5R)-5-{[(5-fluoropyridin-2-yl)oxy]methyl}-2-methylpiperidin -1-yl)carbonyl]-4-methylphenyl}pyrimidine (filorexant), which is shown as Compound M:

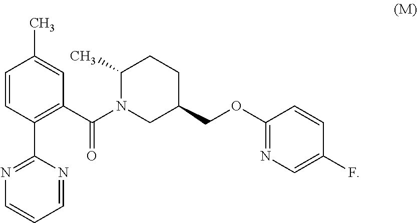 [SMILES: Cc1ccc(-c2ncccn2)c(C(=O)N2C[C@H](COc3ccc(F)cn3)CC[C@H]2C)c1]

In embodiments, the one or more API or pharmaceutically acceptable salt thereof, is present in a concentration of from about 0.1% w/w to about 40% w/w. In particular instances, the one or more API, or a pharmaceutically acceptable salt thereof, is present in a concentration of from about 5% w/w to about 35% w/w, or from about 10% w/w to about 30% w/w. All other variables are as provided above.

The one or more API may be in the form of a pharmaceutically acceptable salt. In instances, the pharmaceutically acceptable salt of the one or more API may be selected from sodium, potassium, calcium, magnesium and quaternary ammonium salts of the one or more API. Additional suitable salts include acid addition salt that may, for example, be formed by mixing a solution of a compound with a solution of a pharmaceutically acceptable acid, such as hydrochloric acid, sulfuric acid, acetic acid, trifluoroacetic acid or benzoic acid.

### Polymer(s)

The one or more pharmaceutically acceptable polymers may enhance the absorption of the API when used in the solid dispersion formulations described herein. The one or more pharmaceutically acceptable polymers are selected from the group consisting of cellulosic polymers, vinyl pyrrolidone polymers and vinyl pyrrolidone/vinyl acetate copolymers.

Cellulosic or cellulose-based polymers include cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate (HPMCAS)). Commercially available examples of these include hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P 55.

The pharmaceutically acceptable polymer may be vinyl pyrrolidone/vinyl acetate copolymers. In particular instances, the pharmaceutically acceptable polymer is copovidone, a copolymer of 1-vinyl-2-pyrrolidone and vinyl acetate in the mass proportion of 3:2. Other useful copolymers contain vinyl pyrrolidone and vinyl acetate in ratios of, for example, 90:10, 80:20, 70:30, and 50:50. The amount of vinyl pyrrolidone can range from about 40% up to about 100%, and the amount of vinyl acetate can range from about 0% up to about 60%. Other vinyl polymers and copolymers having substituents that are hydroxy, alkyl, acyloxy, or cyclic amides include polyethylene polyvinyl alcohol copolymers; and polyvinyl caprolactam-polyvinyl acetate-polyethylene glycol graft copolymer (such as those commercially available as SOLUPLUS®, BASF Corp.). Commercially available copolymers of vinyl pyrrolidone and vinyl acetate include PLASDONE® S630 (Ashland, Inc., Covonton, Ky.) and KOLLIDON® VA 64 (BASF Corp., Florham Park, N.J.), which contain vinyl pyrrolidone and vinyl acetate in a 60:40 ratio. Other copolymers of vinyl pyrrolidone and vinyl acetate can also be used in the invention. In particular instances, the copolymer contains at least 40% vinyl pyrrolidone, although smaller amounts of vinyl pyrrolidone can also be utilized.

The one or more pharmaceutically acceptable polymers are present in a concentration of from about 1% w/w to about 90% w/w. In particular instances, the one or more pharmaceutically acceptable polymers are present in a concentration of from about 10% w/w to about 70% w/w, or about 65% w/w. All other variables are as provided above.

### Optional Surfactant(s)

The action of polymers may be improved by the presence of one or more pharmaceutically acceptable surfactants. The surfactants can increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Surfactants may also facilitate absorption of APIs by altering API permeability and/or efflux directly. See, e.g., Lawrence Yu et al., Vitamin E-TPGS Increases Absorption Flux of an HIV Protease Inhibitor by Enhancing Its Solublity and Permeability, 16(12) PHARM. RES. 1812-1817 (1999). Non-limiting examples of pharmaceutically acceptable surfactants that are suitable for the present invention include polyoxyethylene castor oil derivates, e.g. polyoxyethyleneglycerol triricinoleate or polyoxyl 35 castor oil (CREMOPHOR® EL; BASF Corp.) or polyoxyethyleneglycerol oxystearate such as polyethylenglycol 40 hydrogenated castor oil (CREMOPHPR® RH 40, also known as polyoxyl 40 hydrogenated castor oil or macrogolglycerol hydroxystearate) or polyethylenglycol 60 hydrogenated castor oil (CREMOPHPR® RH 60); or polysorbates or mono fatty acid esters of polyoxyethylene sorbitan, such as a mono fatty acid ester of polyoxyethylene (20) sorbitan, e.g. polyoxyethylene (20) sorbitan monooleate (polysorbate 80, commercially available as TWEEN® 80), polyoxyethylene (20) sorbitan monostearate (polysorbate 60, commercially available as TWEEN® 60), polyoxyethylene (20) sorbitan monopalmitate (polysorbate 40, commercially available as TWEEN®40), or polyoxyethylene (20) sorbitan monolaurate (polysorbate 20, commercially available as TWEEN®20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate; sorbitan fatty acid mono esters such as sorbitan mono laurate (such as those commercially available as SPAN® 20), sorbitan monooleate, sorbitan monopalnitate (such as those commercially available as SPAN® 40), or sorbitan stearate; D-alpha-tocopheryl polyethylene glycol 1000 succinate (vitamin E TPGS); or a combination or mixture thereof. Other non-limiting examples of suitable surfactants include anionic surfactants, e.g. docusate potassium, docusate sodium, docusate calcium and sodium lauryl sulfate (SLS). Other suitable surfactants include, but are not limited to, block copolymers of ethylene oxide and propylene oxide, also known as polyoxyethylene polyoxypropylene block copolymers or polyoxyethylene polypropyleneglycol, such as those commercially available as POLOXAMER® 124, POLOXAMER® 188, POLOXAMER® 237, POLOXAMER® 388, or POLOXAMER® 407 (BASF Corp.). As described above, a mixture of surfactants can be used in a solid composition of the present invention. In particular instances, the surfactant is selected from the group consisting of SLS, vitamin E TPGS, or nonionic ethoxylated alcohols like polysorbate or poloxamer. In particular instances, the surfactant is selected from SLS and vitamin E TPGS. All other variables are as provided above.

The one or more pharmaceutically acceptable surfactant may be present in a concentration of from about 2% w/w to about 20% w/w. In particular instances, the one or more pharmaceutically acceptable surfactant is present in a concentration of from about 3% w/w to about 10% w/w, or about 5% w/w. All other variables are as provided above.

### Solid Dispersion Formation

The solid dispersion formulations described herein relate to solid dispersion formulations produced by solvent removal (e.g., spray drying), introduction of an antisolvent (e.g., precipitation), addition of heat together with mixing (e.g., extrusion), mechanical activation or other means (e.g., to produce a “solid dispersion intermediate”). That is, the solid dispersion formulation may be formed by a process selected from a method selected from lyophilization, film casting, co-precipitated amorphous dispersion (cPAD), extrusion methods such as hot-melt extrusion, spray drying, melting method, solvent evaporation method, fusion method, kneading method, melting method, co-grinding method, melt agglomeration, and supercritical fluid (SCF) technology. See Ladan Akbarpour Nikghalb et al., Solid Dispersion: Methods and Polymers to Increase the Solubility of Poorly Soluble Drugs, 2(10) J. APP. PHARM. SCI. 170-175 (2012). In particular instances, the solid dispersion formulation comprises particles of the composition formed by spray drying or hot melt extrusion.

Spray drying is well known (see, e.g., Masters, Spray Drying Handbook, 1991, 5^{th} edition, Longman Scientific & Technical) and widely practiced in a variety of industrial applications including spray drying of milk (see, e.g., U.S. Pat. No. 4,187,617) and pharmaceutical products (see, e.g., U.S. Pat. No. 6,763,607). In spray drying, the polymer, drug, and optional surfactant, are dissolved in a solvent and then are sprayed through a nozzle as a fine spray into a chamber where the solvent is evaporated quickly to make particles comprising polymer, drug, and optional surfactant. Ideally, the solvent is any solvent in which all of the components of the composition are soluble and that is readily evaporated in a spray dryer. The solvent should also be suitable for use in preparing pharmaceutical compositions. In certain embodiments of the invention, the use of mixed-solvent systems, particularly those containing a combination of water and another solvent, are necessary to facilitate the production of solid solution intermediates containing drug, polymer or polymer(s), and, optionally a surfactant.

Useful solvents for spray drying include water, acetone, ethanol, methanol, dichloromethane, isopropanol and tetrahydrofuran (THF). The spray drying may be performed in a mixed-solvent system. A mixed-solvent system is a solvent system that comprises a first solvent and a second solvent. In aspects, the first solvent may be selected from the group consisting of acetone, ethanol, methanol, dichloromethane, isopropanol and tetrahydrofuran (THF); the second solvent is water. In particular aspects, the first solvent may be selected from the group consisting of ethanol, methanol and acetone; the second solvent is water. In specific instances, the first solvent is acetone and the second solvent is water. The proportions of the first solvent to second solvent may be about 90:10. Mixed-solvent systems are described in PCT International Patent Application Publication No. WO2007/109605 and U.S. Patent Application Publication No. US2007/0026083. Solids loading, which usually refers to the concentration of solid components in the spray-drying solvent system, does not typically exceed 50% and depends on solution properties, such as solubility, stability and viscosity. The solids, comprising API, the pharmaceutically acceptable polymer and any optional surfactant, are present in the spray drying solution in a concentration of from about 5% w/w to about 50% w/w, based on the solubility, stability and viscosity of the solution. In particular instances, the solids are present in the solution in a concentration of from about 10% w/w to about 30% w/w.

Following formation of a solid dispersion formulation, the resulting spray-dried intermediate can undergo a secondary drying step to remove residual solvents. This secondary drying unit operation can occur in a static dryer or agitated dryer. Gas, humidified gas, or vacuum may be applied to the material in the secondary dryer and such application can be useful in more rapidly removing residual solvents that remain in the spray-dried intermediate. See, e.g.,

European Patent Application Publication No. EP1855652 A2 (and references therein) and PCT International Patent Application Publication No. WO2008/012617A1 (and references therein).

In hot melt extrusion, the polymer, drug, and optional surfactant may be either premixed together (e.g., via a wet granulation process) or fed as independent feed streams into the extruder (see Polymer Extrusion 4^{th} Edition by Chris Rauwendaal 2001, Hanser Gardner Publications, Inc., Cincinnati, Ohio or Schenck et al., (2010), Achieving a Hot Melt Extrusion Design Space for the Production of Solid Solutions, in Chemical Engineering in the Pharmaceutical Industry: R&D to Manufacturing (ed. D. J. am Ende), John Wiley & Sons, Inc., Hoboken, N.J., USA). In accordance with this embodiment, any means for preparing a melt in any convenient apparatus in which an admixture of API, polymer and, optionally a surfactant can be heated and optionally mixed can be used. Solidification can be carried out by merely cooling the melt. Once a solid is obtained, the solid can be further mechanically processed to provide a convenient form for incorporation into a medicament, for example, tablets or capsules.

It will be appreciated that other methods of preparing a melt, solidifying it, and forming the solid into conveniently sized particles can be utilized without departing from the spirit of the invention. For example, compositions of the invention may be prepared using an extruder. When an extruder is employed to prepare compositions of the invention, the material may be introduced into the extruder either in a pre-flux state, that is, as a dry admixture, or in a fluxed state, that is in a melted, plastic, or semi-solid state achieved after the application of sufficient heat to the admixture to cause the API to dissolve in the polymer, optionally when a fluxed charge is prepared, blending may be employed during heating to promote uniformity of the fluxed material.

If the material is introduced to the extruder in a fluxed state, residence time in the extruder is selected to be just sufficient to ensure homogeneity of the composition and the temperature is preferably maintained in the extruder at a level just sufficient to insure that the material maintains its plasticity so that it can be extruded into a conveniently shaped extrudate. If the material is introduced into an extruder in a pre-flux state, the extruder components, for example, the barrels and any mixing chamber present in the equipment, will be maintained at a temperature sufficient to promote fluxing of the admixture. Temperatures selected for use in processing a composition will also take into account that blending which occurs within the extruder equipment, for example, in a mixing section of the barrels, will also contribute to localized fluxing of the admixture by imparting shear-stresses that induce heating in the mixture.

Additionally it will be appreciated that equipment temperatures and residence times will be selected to minimize the amount of time that the admixture placed into the extruder spends under conditions of heating and/or shear stress so as to minimize the amount of API, which may be decomposed during formation of the composition, as discussed above. In general, extrusion processes in which heating is applied to the material extruded are termed “hot melt extrusion processes.” When compositions of the present invention are prepared using extrusion equipment, the extrudate thus provided can be in any convenient shape, for example, noodles, cylinders, bars, or the like. If desired, the extrudate can be further processed, for example by milling, to provide a particulate form of the composition.

Co-precipitated amorphous dispersion (cPAD) occurs where the polymer, API, and optional surfactant are dissolved in a solvent or mixture of solvents and precipitated together in a non-solvent to produce the amorphous dispersion. See Navnit Shah et al., Development of Novel Microprecipitated Bulk Powder (MBP) Technology for Manufacturing Stable Amorphous Formulations of Poorly Soluble Drugs, 438 INTL J. PHARM. 53-60 (2012).

Lyophilization (freeze drying) occurs where the polymer, API, and optional surfactant are dissolved in a solvent or mixture of solvents and subsequently frozen and low temperature. The frozen solid is placed under vacuum where the frozen solvent is sublimed leaving behind the amorphous dispersion. See Farzana S. Bandarkar & Ibrahim S. Khattab, Lyophilized Gliclazide-poloxamer Solid Dispersions for Enhancement of In-Vitro Dissolution and In-Vivo Bioavailability, 3(Supp. 2) INTL. J. PHARMACY AND PHARM. SCI. 122-127 (2011).

Solvent evaporation method/film-casting occurs where the polymer, API, and optional surfactant are dissolved in a solvent or mixture of solvents and the solvent is allowed to to evaporate at ambient temperature pressures or under the assistance of elevated temperature and vacuum to give the amorphous solid dispersion. See S. Sethia & E. Squillante, Solid Dispersion of Carbamazepine in PVP K30 by Conventional Solvent Evaporation and Supercritical Methods, 272 INTL J. PHARM. 1-10 (2004).

### Granulation Intermediate

The solid dispersion formulation(s) may be granulated to form a granulation intermediate. The granulation intermediate may be produced by methods known in the art, including compaction, high shear (wet or dry) and roller milling, as well as by any known or later discovered methods of preparing granulates. In embodiments in which two or more solid dispersion formulations are employed, the solid dispersion formulations may be granulated individually or co-granulated. In additional embodiments, the one or more solid dispersion formulation(s) may be co-granulated with one or more crystalline APIs to produce the granulation intermediate.

Optionally, the granulation intermediate may contain a disintegrant or the disintegration system as described above. The disintegration system may be present in the granulation intermediate in an amount from about 6% to about 30% by weight of the total granulation intermediate.

Particular embodiments provide granulation intermediates, comprising a) one or more solid dispersion formulations, each independently comprising i) one or more pharmaceutical active ingredients, ii) one or more pharmaceutically acceptable polymers, and iii) optionally one or more pharmaceutically acceptable surfactants, and wherein said one or more pharmaceutical active ingredients and said one or more optional surfactants are dispersed in a polymer matrix formed by said one or more pharmaceutically acceptable polymers and wherein the one or more solid dispersion formulations are each particulates; and b) a disintegrant selected from the group consisting of modified starches, cross-linked polyvinylpyrrolidones, modified celluloses, soy polysaccharides, cross-linked alginic acids, gellan gum, xantham gum, calcium silicate and ion exchange resins; and c) an inorganic salt, where the inorganic salt is in the form of particles, wherein said particles are characterized by (i) a d₅₀ value of less than about 325 micron; (ii) a d₁₀ value of less than about 185 micron; and (iii) a d₉₀ value of less than about 470 micron; wherein the disintegrant and the inorganic salt are provided in a ratio of from about 2:1 to about 1:3. All variables are as provided above.

### Blended Compositions

Embodiments of the invention relate to blended compositions that comprise a) one or more solid dispersion formulations, as previously described, and a disintegration system, as previously described. In particular embodiments, the salt of the disintegration system and the disintegrant of the disintegration system are added individually to the blend to form the blended composition. In alternative embodiments, the salt of the disintegration system and the disintegrant of the disintegration system may be mixed together prior to being blended with the solid dispersion formulations.

Embodiments of the invention relate to blended compositions that comprise a) one or more granulation intermediates, as previously described, and a disintegration system, as previously described. In particular embodiments, the salt of the disintegration system and the disintegrant of the disintegration system are added individually to the blend to form the blended composition. In alternative embodiments, the salt of the disintegration system and the disintegrant of the disintegration system may be mixed together prior to being blended with the granulation intermediates.

Embodiments of the invention relate to blended compositions that comprise a) one or more solid dispersion formulations, as previously described, one or more crystalline APIs and a disintegration system, as previously described. In particular embodiments, the salt of the disintegration system and the disintegrant of the disintegration system are added individually to the blend to form the blended composition. In alternative embodiments, the salt of the disintegration system and the disintegrant of the disintegration system may be mixed together prior to being blended with the solid dispersion formulations.

Embodiments of the invention relate to blended compositions that comprise a) one or more granulation intermediates, as previously described, one or more crystalline APIs and a disintegration system, as previously described. In particular embodiments, the salt of the disintegration system and the disintegrant of the disintegration system are added individually to the blend to form the blended composition. In alternative embodiments, the salt of the disintegration system and the disintegrant of the disintegration system may be mixed together prior to being blended with the granulation intermediates.

In all embodiments of the blended compositions, all variables with respect to the solid dispersion formulations are as provided above.

In embodiments, the solid dispersion formulation is present in the blended composition in a concentration of from about 3% w/w to about 75% w/w. In particular instances, the solid dispersion formulation is present in the blended composition in a concentration of from about 10% w/w to about 55% w/w, or about 20% w/w to about 40% w/w.

In embodiments, the disintegant system is present in the blended composition in a concentration of from about 3% w/w to about 45% w/w. In particular instances, the disintegrant system is present in the blended composition in a concentration of from about 6% w/w to about 30% w/w, or about 20% w/w.

In embodiments, the granulation intermediate is present in the blended composition in a concentration of from about 3% w/w to about 100% w/w. In particular instances, the granulation intermediate is present in the blended composition in a concentration of from about 10% w/w to about 75% w/w, or about 20% w/w to about 50% w/w.

In embodiments, one or more diluents may be present in the blended composition. A “diluent” is an excipient which increases the bulk of a dosage form, typically where the active pharmaceutical ingredient in the formulation is too potent to permit convenient processing or administration of a dosage form that does not include a diluent, or where the formulation by itself without a diluent makes formation of the dosage form difficult (for example, where an aliquot of the formulation without a diluent would be of too small of a volume to form the aliquot into a tablet). The diluent in the blended composition may be one or more pharmaceutically acceptable diluents selected from the group consisting of mannitol, microcrystalline cellulose, calcium carbonate, sodium carbonate, lactose, dicalcium phosphate, sodium phosphate and kaolin, and combinations thereof. In particular aspects, the diluent is one or more selected from the group consisting of lactose, microcrystalline cellulose, mannitol and dicalcium phosphate. In a particular instance, the diluent is a combination of lactose, mannitol and microcrystalline cellulose. In particular embodiments, the diluent is present in the blended composition in a concentration of from about 3% w/w to about 58% w/w. In particular instances, the diluent is present in a concentration of from about 18% w/w to about 50% w/w, or about 38% w/w.

In embodiments, one or more additional disintegrants may be present in the blended composition. The additional disintegrant(s) may be chosen from conventional disintegrants and/or from the disintegrants listed above. In particular embodiments, the disintegrant in the blended composition is selected from the group consisting of croscarmellose sodium, sodium starch glycolate and crospovidone. In particular instances, the disintegrant is croscarmellose sodium. In embodiments, the disintegrant is present in the blended composition in a concentration of from about 5% w/w to about 20% w/w. In particular instances, the disintegrant is present in a concentration of from about 6% w/w to about 15% w/w, or about 10% w/w.

In embodiments, one or more additional salts may be present in the blended composition. The additional salt is selected from the group consisting of NaCl, KCl, CaCl₂, KH₂PO₄, NaH₂PO₄, K₂SO₄, NaHCO₃, K₂CO₃ and combinations thereof. In aspects, the additional salt in the blended composition is selected from the group consisting of NaCl, KCl, CaCl₂ and combinations thereof. In a particular instance, the additional salt is NaCl. In particular embodiments, the salt is present in the blended composition in a concentration of from about 5% w/w to about 20% w/w. In particular instances, the salt is present in a concentration of from about 6% w/w to about 15% w/w, or about 10% w/w.

In embodiments, one or more lubricants may be present in the blended composition. A “lubricant” is an excipient that reduces friction in a formulation and allows for improved flow and improved processability, primarily by reducing friction between the tablet surface and the die wall and reducing sticking of the formulation to tablet punch surfaces. The lubricant in the blended composition may be selected from the group consisting of magnesium stearate and sodium stearyl fumarate or both. In embodiments, the lubricant is present in the blended composition in a concentration of from about 0.5% w/w to about 5% w/w. In particular instances, the lubricant is present in a concentration of from about 1% w/w to about 3% w/w.

In embodiments, one or more glidants may be present in the blended composition. A “glidant” is an excipient, for example colloidal silica, that enhances the flow of a granular mixture by reducing interparticle friction. The glidant in the blended composition may be selected from the group consisting of starch, talc, magnesium stearate and silicon dioxide (SiO₂, including colloidal silicon dioxide, silicon dioxide, fumed silica, pyrogenic silica, and those commercially available as CAB O SIL® (Cabot), AEROSIL®, SIPERNAT®, and SIDENT® (Evonik) and combinations thereof. In a particular instance, the glidant is silicon dioxide. In embodiments, the glidant is present in the blended composition in a concentration of from about 0% w/w to about 2.5% w/w. In particular instances, the glidant is present in a concentration of from about 0.1% w/w to about 1% w/w, or about 0.5% w/w.

In embodiments, the blended composition may also include one or more additional excipients selected from the group consisting of sweeteners or sweetening agents, flavoring agents, colorants or coloring agents, preservatives or preserving agents, binders or binding agents, and antioxidants. Such excipients may be used as known and understood in the art.

A further embodiment is directed to a process for preparing a blended composition comprising the steps of: a) preparing a solid dispersion formulation comprising an API or a pharmaceutically acceptable salt thereof, ii) blending the solid dispersion formulation with a disintegration system as described above, and iii) optionally granulating to produce a granulation intermediate; b) mixing the product of step a) and optionally one or more of a diluent, disintegrant, salt, lubricant and glidant together; and c) optionally granulating the blend of step c) to produce a blended composition. In aspects of this embodiment, blending may comprise blending alone, blending followed by granulation, or granulation followed by blending with excipients. Granulation, as used herein, includes all known and later-developed methods of creating granulation.

In aspects of these embodiments, the diluents, disintegrants, salts, lubricants and/or glidants are as described above. The diluents, disintegrants, salts, lubricants and/or glidants may be present in the concentrations described above.

### Oral Dosage Forms

In embodiments, pharmaceutical oral dosage form, comprising a) one or more solid dispersion formulations, each independently comprising i) one or more active pharmaceutical ingredients, ii) one or more pharmaceutically acceptable polymers, and iii) optionally one or more pharmaceutically acceptable surfactants, and wherein said one or more active pharmaceutical ingredients and said one or more optional surfactants are dispersed in a polymer matrix formed by said one or more pharmaceutically acceptable polymers; and b) a disintegrant selected from the group consisting of modified starches, cross-linked polyvinylpyrrolidones, modified celluloses, soy polysaccharides, cross-linked alginic acids, gellan gum, xantham gum, calcium silicate and ion exchange resins; and c) an inorganic salt, where the inorganic salt is in the form of particles, wherein said particles are characterized by (i) a d₅₀ value of less than about 325 micron; (ii) a d₁₀ value of less than about 185 micron; and (iii) a d₉₀ value of less than about 470 micron; d) optionally one or more additional active pharmaceutical ingredients; and e) optionally one or more excipients selected from the group consisting of diluents, additional disintegrants, additional salts, lubricants, glidants, sweetening agents, flavoring agents, coloring agents, preserving agents, binding agents, and antioxidants; wherein the disintegrant and the inorganic salt are provided in a ratio of from about 2:1 to about 1:3; and wherein said pharmaceutical oral dosage form is a tablet or a capsule.

In embodiments of the pharmaceutical oral dosage forms described herein, the one or more solid dispersion formulations, disintegrant and inorganic salt are in the form of a granulation intermediate.

In embodiments of the pharmaceutical oral dosage forms described herein, the one or more solid dispersion formulations, disintegrant and inorganic salt are in the form of a blended composition. In particular instances, the blended composition comprises the one or more solid dispersion formulations, disintegrant and inorganic salt and any additional API and any additional excipients.

An additional embodiment of the invention is directed to a process for preparing a solid pharmaceutical composition comprising the steps of: a) preparing a blended composition as described above in embodiments; and b) compressing the blended composition into a tablet or filling into a capsule. In aspects of this embodiment, the tablet is optionally film-coated; in further aspects, the tablet or capsule is optionally photo-shielded, for example by use of a blister packaging.

In aspects of these embodiments, the diluents, disintegrants, salts, lubricants and/or glidants are as described above with respect to blended compositions. The diluents, disintegrants, salts, lubricants and/or glidants may be present in the concentrations described above with respect to blended compositions.

Tablets may contain the active ingredient in admixture with non-toxic pharmaceutically acceptable excipients that are suitable for the manufacture of tablets. These excipients may be for example, inert diluents, granulating and disintegrating agents, binding agents, glidants, lubricating agents, and antioxidants, for example, propyl gallate, butylated hydroxyanisole and butylated hydroxy toluene. The tablets may be uncoated or they may be coated to delay disintegration and absorption in the gastrointestinal tract and thereby provide a sustained action over a longer period.

Compositions for oral use may also be presented as capsules (e.g., hard gelatin) wherein the active ingredient is mixed with an inert solid diluent, for example, calcium carbonate, calcium phosphate or kaolin, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent and one or more preservatives. In certain embodiments of the invention, the pharmaceutical compositions of the invention include a diluent system, lubricant, glidant and filmcoat, at concentrations of as described above with respect to blended compositions. In certain embodiments, the solid dispersion formulations are blended with a diluent, one or more disintegrating agents, lubricant and glidant. An exemplary blended composition or oral dosage form includes mannitol, microcrystalline cellulose, croscarmellose sodium, sodium chloride, colloidal silica and magnesium stearate.

The additional disintegrant may be present in a concentration from about 5% w/w to about 20% w/w or from about 6% w/w to about 15% w/w. A salt may be also present, which may be sodium chloride, potassium chloride or a combination thereof. The combination of salts and additional disintegrant is present at a concentration from about 10% w/w to about 30% w/w of the final pharmaceutical composition. Pharmaceutical compositions comprising these levels of disintegrant and salt (in combination with polymer(s)) provide a rapidly disintegrating dosage form. Rapidly disintegrating tablets based on solid dispersion formulations are disclosed in U.S. Pat. No. 7,189,415.

The blended compositions may be roller compacted or wet granulated to densify and/or reduce the risk of segregation of components during subsequent handling (e.g., compression into tablets). Granulation steps can also be used to minimize the impact of raw material property variability (e.g., excipient particle size) on subsequent processing (e.g., tablet compression) and ultimate product performance. Lubrication is typically performed prior to roller compaction and tablet compression to reduce the tendency of material to adhere to compression surfaces (e.g., tablet tooling). In particular embodiments, the lubricant is magnesium stearate. These methods can be carried out by those skilled in the art. See, e.g., Ansel, Introduction to Pharmaceutical Dosage Forms, Seventh Edition, 1999.

To prepare the pharmaceutical compositions of the invention, the solid dispersion formulation or blended composition is compressed into an oral dosage form including tablets or capsules. Tablets can be prepared with a variety of possible shapes (ellipsoidal, capsule, biconvex round, etc.). The powder can also be encapsulated in capsule dosage (e.g., using hard gelatin capsules). Techniques suitable for preparing solid oral dosage forms of the present invention are described in Remington's Pharmaceutical Sciences, 18th edition, edited by A. R. Gennaro, 1990, Chapter 89 and in Remington—The Science and Practice of Pharmacy, 21st edition, 2005, Chapter 45.

### EXAMPLES

The following examples serve only to illustrate the invention and its practice. The examples are not to be construed as limitations on the scope or spirit of the invention.

In addition, the following abbreviations are used throughout this specification and in the Examples. Each of these terms has the meaning listed below.

### Abbreviations

% w/w Percent by weight
BHA Butylated hydroxyanisole
BHT Butylated hydroxy toluene
EG Extragranular
g gram
g/min. Grams per minute
h Hour(s)
h:mm:ss Hours:minutes:seconds
HCl Hydrogen chloride
HME Hot melt extrudate
HPMC Hydroxypropylmethyl cellulose
HPMCAS Hydroxypropylmethylcellulose acetate succinate
IG Intragranular
in. ″ Inch
INT Intermediate
KN, kN Kilonewton
kP Kilopond, a non-standard gravitational unit of force, also kilogram-force; 1 kp=9.80665 Newtons
L Liter
mg Milligram
min Minute(s)
mL Milliliter
mm Millimeter
mm:ss Minutes:seconds
MPa Megapascal
NaCC Croscarmellose sodium
NaCl Sodium chloride
PSI, psi Pounds per square inch
rpm Revolutions per minute
RSC Round standard concave (tooling)
s Second(s)
SDI Spray dried intermediate
SGF Simulated Gastric Fluid (1 L water, 1.4 mL concentrated 0.1 N HCl, 2.0 g NaCl, pH 1.8)
SiO₂ Silicon dioxide
SLS Sodium lauryl sulfate
TS Tensile strength
Um, μ Micron, micrometer
USP U.S. Pharmacopeial Convention
Vitamin E TPGS Vitamin E polyethylene glycol succinate

### Example 1

Compound A, copovidone and SLS was dissolved in a 9:1 acetone:water solvent mixture at a solids loading of 7%. The solids were comprised of 30% Compound A, 65% copovidone and 5% SLS. The resulting solution was spray dried on an SD MICRO™ spray dryer and secondary dried in a vacuum oven. The spray dried dispersion was blended with the intragranular components shown in Table 1 on a laboratory mixer (TURBULA®) for 5 min. at 46 rpm followed by lubrication with magnesium stearate for 2 min. at 46 rpm. The resultant blend was slugged on an MTS compression machine at a slugging force of 6.5 KN using ¾ inch knurled tooling to produce slugs of approximately 2 mm in thickness. Granules were formed by pushing the slugs through a 1 mm mesh screen and blended with extragranular magnesium stearate for 5 min. on a laboratory mixer (TURBULA®) at 46 rpm. Tablets of 1.0 gram weight were compressed at 70 MPa compaction pressure on the MTS compression machine using 10/32″× 24/32″ modified oval tooling. The disintegration time of the resulting tablets was measured using a standard USP reciprocating disintegration apparatus with cylinders in 900 mL of SGF at 37° C. Surprisingly, tablets containing both salt (NaCl or KCl) and croscarmellose sodium had the fastest disintegration times as shown in Table 1.

TABLE 1
Disintegration times for Oral Dosage Forms of Compound A

| | | All | All | Disintegrant/ | Disintegrant/ | All |
|---|---|---|---|---|---|---|
| | | Disintegrant | Salt | Salt | Salt | Together |
| Component | Function | w/w % | w/w % | w/w % | w/w % | w/w % |
| Solid | Active | 33.33 | 33.33 | 33.33 | 33.33 | 33.33 |
| Dispersion | | | | | | |
| Intermediate | | | | | | |
| Mannitol | Diluent | 44.92 | 44.92 | 44.92 | 44.92 | 24.92 |
| Sodium Starch | Super | 10 | | | | 10 |
| Glycolate | Disintegrant | | | | | |
| Croscarmellose | Super | 10 | | 10 | 10 | 10 |
| Sodium | Disintegrant | | | | | |
| KCl | Salt | | 10 | | 10 | 10 |
| NaCl, Powder | Salt | | 10 | 10 | | 10 |
| Colloidal | Glidant | 0.25 | 0.25 | 0.25 | 0.25 | 0.25 |
| Silicon Dioxide | | | | | | |
| Magnesium | Lubricant | 1 | 1 | 1 | 1 | 1 |
| Stearate | | | | | | |
| (non bovine) | | | | | | |
| intra | | | | | | |
| Magnesium | Lubricant | 0.5 | 0.5 | 0.5 | 0.5 | 0.5 |
| Stearate | | | | | | |
| (non bovine) | | | | | | |
| intra | | | | | | |
| Total | | 100 | 100 | 100 | 100 | 100 |
| Average | | 25:50 | >30 | 5:56 | 5:33 | <5 |
| Disintegration | | | | | | |
| Time (mm:ss) | | | | | | |

### Example 2

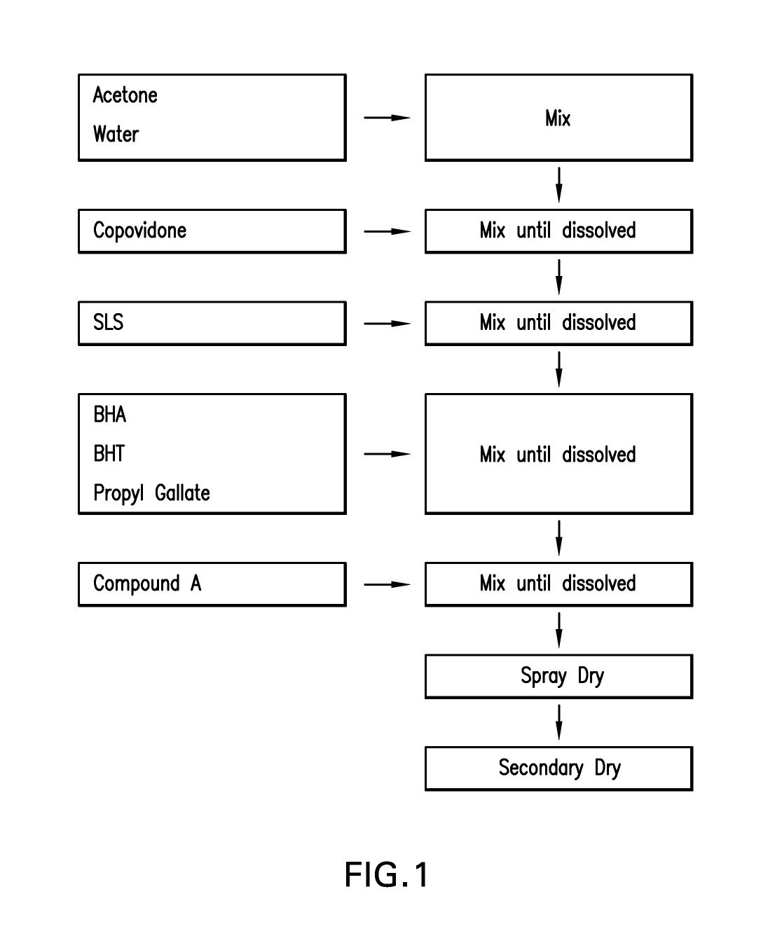

Formulations were prepared from solid solutions of Compound A, SLS, and copovidone by spray drying from a 90/10 (w/w) acetone/water solvent system in a process as described by FIG. 1. The three solid components of the spray drying solution were incorporated into the solution at 20% w/w. A NIRO PSD-2 spray dryer with a pressure nozzle was used to produce the spray dried particles. Heated nitrogen was supplied to the spray dryer at an inlet temperature sufficient to maintain a 52° C. outlet temperature and a gas flow rate of 7500 g/min. The spray drying solution flow rate was 700-800 g/min, which required a nozzle pressure of approximately 400 PSI.

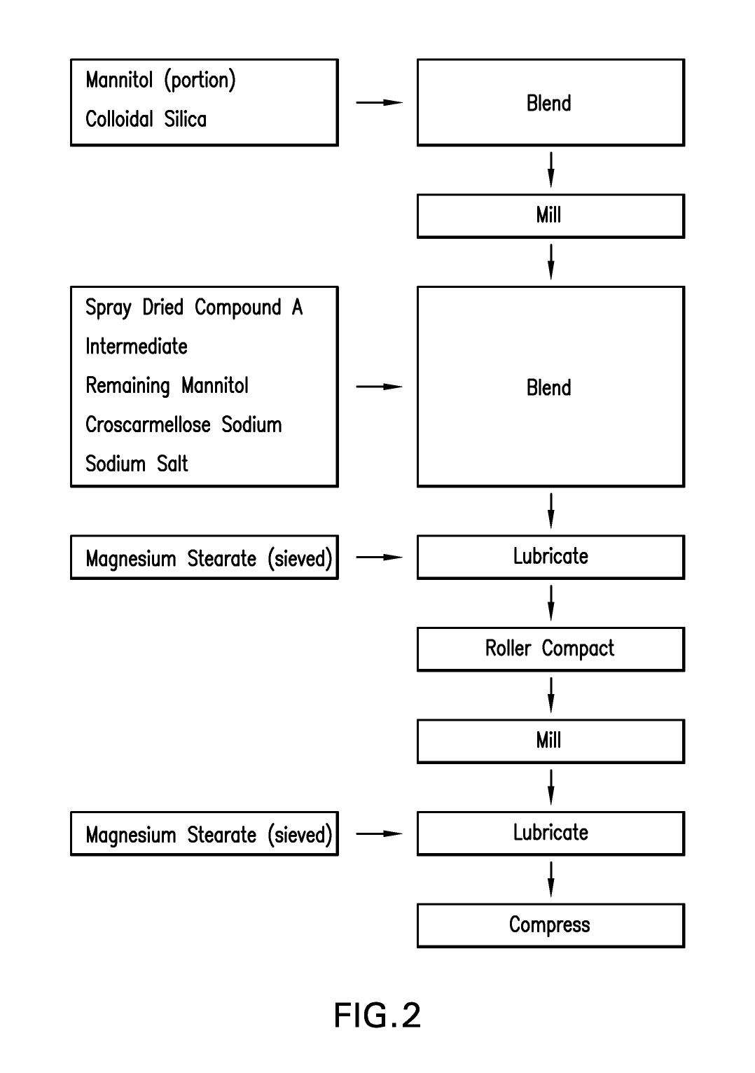

A tablet composition (formulation 2a) was prepared with a composition identical to that described in Table 2 and using a similar process as illustrated in FIG. 2, but resulting in a tablet of half the size (500 mg vs. 1000 mg). Additional tablet compositions (Formulations 2b-2e) were prepared using different levels and types of salt (NaCl, KCl, CaCl₂, etc.), grades of salt (coarse vs. fine, mean particle size of approximately 380 μm and 185 μm, respectively), and disintegrant (croscarmellose sodium, sodium starch glycolate, etc.). A master blend of the spray dried intermediate, mannitol and colloidal silica was prepared by co-sieving materials through a

No. 30 mesh and blending using a laboratory mixer (TURBULA®) for 5 min. at 46 RPM. Appropriate amounts of the relevant disintegrant and/or salt were sieved through a No. 30 mesh and blended with a portion of the master blend using a laboratory mixer (TURBULA®) for 5 min. at 46 RPM. Magnesium stearate (sieved through No. 60 mesh) was added to the blends and the mixture was lubricated using a laboratory mixer (TURBULA®) for 2 minutes at 46 RPM. The tablet compositions were made by compressing the powder blend containing the spray dried solid dispersion into tablets using a 16/32″ round standard concave tooling on small scale single station compression equipment (Carver, MTS or Lloyds). The tablet hardness measurements are listed in Table 3. The disintegration time of the resulting tablet compositions was measured using a standard USP reciprocating disintegration apparatus with cylinders in 900 mL of SGF at 37° C. Table 3 summarizes the measured disintegration behavior of these formulations compared with the reference (Formulation 2a).

TABLE 2
Composition of Formulations of Example 2

| | | | | | |
|---|---|---|---|---|---|
| | Formulation | Formulation | Formulation | Formulation | Formulation |
| Components | 2a (mg/tab) | 2b (mg/tab) | 2c (mg/tab) | 2d (mg/tab) | 2e (mg/tab) |

Spray Dried Intermediate

| | | | | | |
|---|---|---|---|---|---|
| Compound A | 50 | 50 | 50 | 50 | 50 |
| Polyvinylpyrolidone/ | 107.1 | 107.1 | 107.1 | 107.1 | 107.1 |
| Vinyl Acetate | | | | | |
| Copolymer | | | | | |
| BHA | 0.4167 | 00.4167 | 0.4167 | 0.4167 | 0.4167 |
| BHT | 0.4167 | 0.4167 | 0.4167 | 0.4167 | 0.4167 |
| Propyl Gallate | 0.4167 | 0.4167 | 0.4167 | 0.4167 | 0.4167 |
| SLS | 8.335 | 8.335 | 8.335 | 8.335 | 8.335 |

Downstream Tablet

| | | | | | |
|---|---|---|---|---|---|
| Mannitol | 224.6 | 224.6 | 224.6 | 224.6 | 224.6 |
| Croscarmellose | 50 | 50 | 50 | 0 | 50 |
| Sodium | | | | | |
| Sodium Starch | 0 | 0 | 0 | 50 | 50 |
| Glycolate | | | | | |
| NaCl (granular) | 50 | 0 | 0 | 0 | 0 |
| NaCl (fine) | 0 | 50 | 0 | 50 | 0 |
| KCl (fine) | 0 | 0 | 50 | 0 | 0 |
| Colloidal Silicon | 1.25 | 1.25 | 1.25 | 1.25 | 1.25 |
| Dioxide | | | | | |
| Magnesium Stearate | 7.5 | 7.5 | 7.5 | 7.5 | 7.5 |
| (non-bovine) | | | | | |
| Total | 500 | 500 | 500 | 500 | 500 |

TABLE 3
Properties of Compositions of Example 2

Disintegration Time (mm:ss)

| | 13:23 | 5:56 | 5:33 | 23:55 | 25:50 |
|---|---|---|---|---|---|
| Compression Force | 10.1 | 10.1 | 10.1 | 11.4 | 10.1 |
| using 16/32″ RSC | | | | | |
| (Target kN) | | | | | |
| Hardness (kP) | 13.9 | 14.7 | 15.3 | 13.7 | 14.9 |
| Calculated Tensile | 1.92 | 2.04 | 2.10 | 1.97 | 1.96 |
| Strength (MPa) | | | | | |

This example demonstrates that combinations of croscarmellose sodium with NaCl or KCl provide observable, enhanced disintegration times.

### Example 3

Compound E, HPMC, and vitamin E TPGS was dissolved in an 7:3 acetone:water solvent system at a 10% solids loading. The solids were comprised of 20% Compound E, 75% HPMC, and 5% vitamin E TPGS. The resulting solution was spray dried on a PSD-1 spray dryer followed by secondary drying, according to U.S. Provisional Patent Application No. 62/095,398 (filed Dec. 22, 2014), Example 2. The tablet composition of Compound E as shown in Table 4 is prepared by blending the spray dried dispersion with the intragranular components in a laboratory mixer (TURBULA®) for 10 min. at 46 rpm followed by lubrication with magnesium stearate in the laboratory mixer (TURBULA®) for 5 min. at 46 rpm. The resultant lubricated blend was slugged on a compaction study machine (by Roland Research Devices Incorporated) using ¾″ knurled tooling at a target tensile strength of 1.0 MPa and slug thickness of approximately 2 mm. Granules were formed by milling the slugs through a 2 mm mesh screen and then a 1 mm mesh screen. The granules were blended with extragranular croscarmellose sodium and magnesium stearate on a laboratory mixer (TURBULA®) for 5 min. at 46 rpm. Tablets of the desired weight were compressed at a target of 100 MPa to 200 MPa compaction pressure on the compaction study machine (by Roland Research Devices Incorporated) using 14/32″ standard round concave tooling to provide tablets with tensile strengths of 1.5 MPa to 2.5 MPa; this procedure is similar to that of U.S. Provisional Patent Application No. 62/095,398 (filed Dec. 22, 2014), Example 3.

TABLE 4
Compositions of Tablets Containing Compound E

| Components | Function | Tablet (mg) | Percent of Tablet |
|---|---|---|---|
| Compound E | Active | 50 | 9 |
| Acetone | Solvent | — | — |
| Purified water | Solvent | — | — |
| Hydroxypropyl | Polymeric | 175 | 31.9 |
| methylcellulose | stabilizer | | |
| (HYPROMELLOSE | | | |
| 2910) | | | |
| Vitamin E | Surfactant | 25 | 4.6 |
| polyethylene glycol | | | |
| succinate | | | |
| Cellulose, | Diluent | 71.25 | 13 |
| microcrystalline | | | |
| Lactose | Diluent | 71.25 | 13 |
| Croscarmellose | Disintegrant | 50 intragranular | 9 intragranular |
| sodium | | 32 extragranular | 6 extragranular |
| NaCl, powder | Disintegrant | 50 | 9 |
| Silicon dioxide, | Glidant | 2.5 | 0.5 |
| colloidal | | | |
| Magnesium stearate | Lubricant | 2.5 intragranular | 0.5 intragranular |
| (non-bovine) | | 2.5 extragranular | 0.5 extragranular |
| Film coat blend, | Film coat | 16 | 3 |
| powder, white | | | |
| (Opadry II | | | |
| 85F18422) | | | |
| Total tablet | — | 548.0 | 100.0 |
| weight | | | |

Formulations containing various levels of salt and intra/extragranular levels of croscarmellose were similarly prepared as shown in Table 5. The disintegration time of the resulting tablets was measured using a standard USP reciprocating disintegration apparatus with cylinders in 900 mL of pH 3.3 achlorohydric media at 37° C. Table 5 illustrates that both disintegrant (croscarmellose sodium) and salt (NaCl) were needed to achieve disintegration times less than 1 h. The use of intragranular (IG) and extragranular (EG) disintegrant is also demonstrated.

TABLE 5
Disintegration Times

| | | | | | | Disintegration |
|---|---|---|---|---|---|---|
| NaCC IG | NaCl | NaCC EG | MPa | Hardness | TS | Time |
| 9 | 0 | 3 | 100 | 16 | 1.6 | 1:46:16 |
| | | | 150 | 19 | 1.9 | 2:01:00 |
| | | | 200 | 24 | 2.4 | 2:00:28 |
| 12 | 10 | 3 | 100 | 13.6 | 1.3 | 9:07 |
| | | | 125 | 16 | 1.6 | 12:42 |
| | | | 200 | 20 | 2.1 | 23:23 |
| 12 | 0 | 6 | 100 | 15 | 1.5 | 1:29:18 |
| | | | 150 | 19 | 1.9 | 1:31:24 |
| | | | 200 | 23.6 | 2.4 | 1:41:53 |
| 6 | 10 | 9 | 150 | 18 | 1.9 | 0:15:39 |
| | | | 200 | 20 | 2.1 | 0:22:34 |
| | | | 250 | 23 | 2.4 | 0:24:55 |
| 9 | 5 | 6 | 100 | 16.6 | 1.6 | 0:23:09 |
| | | | 150 | 20 | 2.1 | 0:34:41 |
| | | | 250 | 23 | 2.3 | 0:40:22 |
| 12 | 5 | 9 | 100 | 16 | 1.6 | 0:28:31 |
| | | | 150 | 19 | 1.9 | 0:33:18 |
| | | | 200 | 23 | 2.4 | 0:38:43 |
| | | | 250 | 24 | 2.5 | 0:42:10 |
| 6 | 0 | 9 | 100 | 17 | 1.7 | 1:38:02 |
| | | | 150 | 20 | 2 | 1:49:42 |
| | | | 200 | 23.5 | 2.4 | 1:59:44 |
| | | | 250 | 25.5 | 2.6 | 2:00:55 |
| 6 | 5 | 3 | 125 | 15 | 1.5 | 0:24:15 |
| | | | 200 | 19 | 2.1 | 0:45:52 |
| | | | 300 | 22 | 2.3 | 1:04:31 |

### Example 4

Granulations of Compound A spray dried dispersion (copovidone-based, as in Example 1 above) and Compound E spray dried dispersion (HPMC-based, as in Example 3) were prepared containing disintegrants and salts (as shown in Table 6) at an approximate mean ribbon tensile strength of about 0.7 MPa to about 1.1 MPa using roller compaction on a WP120 roller compactor. Granules of Compound H spray dried intermediate (1:2 Compound H:HPMC-AS) were prepared by blending the Compound H spray dried intermediate with the excipients in Table 6 in a laboratory mixer (TURBULA®) for 10 min. at 46 rpm followed by lubrication with magnesium stearate in a laboratory mixer (TURBULA®) for 2 min. at 46 rpm. Slugs approximately 2 mm in thickness at an approximate mean tensile strength of about 0.7 MPa to about 1.1 MPa were prepared using ¾″ knurled tooling on a compaction study machine (by Roland Research Devices Incorporated). Granules were produced by milling the slugs through a 1 mm screen. Compound H granules were then blended with Compound A granules, Compound E granules, and extra-granular excipients and lubricated in a laboratory mixer (TURBULA®) for 10 min. and 2 min., respectively, prior to compression. Tablets were compressed using 21.2 mm×11.9 mm modified oval tooling at a total tablet weight of 1500 mg and compaction pressure of about 100 MPa. The disintegration time of the resulting tablets was measured using a standard

USP reciprocating disintegration apparatus with cylinders in 900 mL of SGF at 37° C. A disintegration time of less than 15 min. was achieved to provide immediate release characteristics.

TABLE 6
Tablets Containing 3 Separate SDI
Granulations & Disintegration System

| | % of | | |
|---|---|---|---|
| Material | Granulation | % of Blend | Tablet (mg) |
| Granulation 1 | | | |
| Compound A SDI | 33.667 | 11.110 | 166.650 |
| Mannitol | 45.374 | 14.973 | 224.600 |
| Croscarmellose Sodium | 10.101 | 3.333 | 50.000 |
| NaCl, Powder | 10.101 | 3.333 | 50.000 |
| Magnesium Stearate | 0.505 | 0.167 | 2.500 |
| SiO₂ | 0.253 | 0.083 | 1.250 |
| Total | 100.000 | 33.000 | 495.000 |
| Granulation 2 | | | |
| Compound E SDI | 50.251 | 8.333 | 125.000 |
| Microcrystalline Cellulose | 14.322 | 2.375 | 35.625 |
| Lactose Monohydrate | 14.322 | 2.375 | 35.625 |
| Croscarmellose Sodium | 10.050 | 1.667 | 25.000 |
| SiO₂ | 0.503 | 0.083 | 1.250 |
| NaCl, Powder | 10.050 | 1.667 | 25.000 |
| Magnesium Stearate | 0.503 | 0.083 | 1.250 |
| Total | 100.000 | 16.583 | 248.750 |
| Granulation 3 | | | |
| Compound H SDI | 67.797 | 30 | 450.000 |
| Microcrystalline Cellulose | 19.209 | 8.5 | 127.500 |
| Croscarmellose Sodium | 11.299 | 5 | 75.000 |
| SiO₂ | 1.130 | 0.5 | 7.500 |
| Magnesium Stearate | 0.565 | 0.25 | 3.750 |
| Total | 100.000 | 44.25 | 663.750 |
| Extra-Granular | | | |
| Magnesium Stearate | — | 0.417 | 6.250 |
| Croscarmellose Sodium | — | 5.000 | 75.000 |
| SiO₂ | — | 0.500 | 7.500 |
| Magnesium Stearate | — | 0.250 | 3.750 |
| Total | — | — | 1500.000 |

This example illustrates the ability to blend multiple dispersions of various polymer types.

### Example 5

Spray dried intermediates of Compound A/copovidone (Example 1), Compound E/HPMC (Example 3) and Compound H/HPMCAS (Example 4) were blended with additional excipients shown in Table 7 in a laboratory mixer (TURBULA®) for 10 min. at 46 rpm. The blend was then lubricated with magnesium stearate in a laboratory mixer (TURBULA®) for 2 min. at 46 rpm. The lubricated blend was slugged on a compaction study machine (by Roland Research Devices Incorporated) with ¾″ knurled tooling to produce slugs with an approximate tensile strength of about 0.7 MPa to about 1.1 MPa and 2 mm thickness. Granules were produced by milling through a 1 mm screen. The granules were then lubricated with magnesium stearate in a laboratory mixer (TURBULA®) for 2 min. at 46 rpm prior to compression on the compaction study machine (by Roland Research Devices Incorporated). Tablets were compressed using 21.2 mm×11.9 mm modified oval tooling at a total weight of 1500 mg at a compaction pressure of approximately 100 MPa. The disintegration time of the resulting tablets was measured using a standard USP reciprocating disintegration apparatus with cylinders in 900 mL of SGF at 37° C. A disintegration time of less than 15 min. was achieved to provide immediate release characteristics.

TABLE 7
Tablets Containing 3 Co-Granulated SDIs and Disintegration System

| Material | % Granulation | % Blend | Tablet (mg) |
|---|---|---|---|
| Granulation | | | |
| Compound A SDI | 11.222 | 11.11 | 166.65 |
| Compound E SDI | 8.418 | 8.33 | 125 |
| Compound H SDI | 30.303 | 30 | 450 |
| Mannitol | 29.098 | 28.81 | 432.1 |
| NaCl, Powder | 10.101 | 10 | 150 |
| Croscarmellose Sodium | 10.101 | 10 | 150 |
| SiO₂ | 0.253 | 0.25 | 3.75 |
| Magnesium Stearate | 0.505 | 0.5 | 7.5 |
| Total | | | 1485 |
| Extra-Granular | | | |
| Magnesium Stearate | | 1 | 15 |
| Total | | | 1500 |

This example illustrates the ability to co-granulate multiple dispersions of various polymer types.

### Example 6

The present example illustrates the application of the dispersion systems described herein to systems containing mixed active systems, wherein one or more of the active ingredients are present as a crystalline drug substance and one or more of the actives are supplied as a spray dried intermediate. As shown in Table 8, spray dried intermediate (Compound A/copovidone from Example 1, Compound E/HPMC from Example 3) and crystalline API (Compound H) were blended with excipients including NaCl and croscarmellose sodium in a laboratory mixer (TURBULA) for 10 min. at 46 rpm followed by lubrication with magnesium stearate in a laboratory mixer (TURBULA®) for 2 min. at 46 rpm to yield a final blend suitable for dry granulation. Dry granulation was performed by slugging the lubricated blend on a compaction study machine (by Roland Research Devices Incorporated) with ¾″ knurled tooling to yield a tensile strength of about 0.6 MPa to about 1.1 MPa and slugs approximately 2 mm in thickness. The slugs were milled through a 1 mm screen and lubricated with magnesium stearate in a laboratory mixer (TURBULA®) for 2 min. at 46 rpm to yield a final blend suitable for compression. Tablets were compressed using 21.2 mm×11.9 mm modified oval tooling at a total weight of 1500 mg at a compaction pressure of approximately 100 MPa. The disintegration time of the resulting tablets was measured using a standard USP reciprocating disintegration apparatus with cylinders in 900 mL of SGF at 37° C. A disintegration time of less than 15 min. was achieved to provide immediate release characteristics.

TABLE 8
Tablets Containing 2 Co-Granulated SDIs,
a Crystalline API & Disintegration System

| | | | Tablet |
|---|---|---|---|
| Material | % Granulation | % Blend | (mg) |
| Granulation | | | |
| Compound A SDI | 11.222 | 11.11 | 166.65 |
| Compound E SDI | 8.418 | 8.333 | 125 |
| Compound H (crystalline) | 13.468 | 13.333 | 200 |
| Microcrystalline Cellulose | 16.835 | 16.667 | 250 |
| Mannitol SD 100 | 29.098 | 28.807 | 432.1 |
| Sodium Chloride, Powder | 10.101 | 10.0 | 150 |
| Croscarmellose Sodium | 10.101 | 10.0 | 150 |
| Colloidal Silicon Dioxide | 0.253 | 0.25 | 3.75 |
| Magnesium Stearate (Non-Bovine) | 0.505 | 0.5 | 7.5 |
| Total | | | 1485 |
| Extra-Granular | | | |
| Magnesium Stearate (Non-Bovine) | | 1 | 15 |
| Total | | | 1500 |

This example illustrates the ability to co-granulate multiple dispersions of various polymer types with additional crystalline API.

### Example 7

Three formulations shown in Table 9, evaluated the use of croscarmellose sodium, crospovidone and sodium starch glycolate as disintegrants. The formulations were prepared by dry granulation and subsequent compression as described in Example 6 and contained Compound A/copovidone solid dispersion, Compound E/HPMC solid dispersion, and crystalline Compound G. Tablets were compressed using 21.2 mm×11.9 mm modified oval tooling at a total weight of 1500 mg at a compaction pressures between 100 MPa and 250 MPa to generate tablets of similar hardnesses across formulations. The disintegration time of the resulting tablets was measured using a standard USP reciprocating disintegration apparatus with cylinders in 900 mL of SGF at 37° C. As shown in Table 10, the mechanical properties of the three formulations under load were equivalent; however, disintegration times were optimum when utilizing the croscarmellose sodium system.

TABLE 9
Tablets Containing Two Co-Granulated SDIs, a Crystalline
API and Disintegration System Using Varying Disintegrants

| | Tablet (mg) | | |
|---|---|---|---|
| Material | FDC02 | FDC06 | FDC07 |
| Granulation | | | |
| Compound A SDI | 333.4 | 333.4 | 333.4 |
| Compound E SDI | 250 | 250 | 250 |
| Compound G (crystalline) | 300 | 300 | 300 |
| Mannitol SD 100 | 290.35 | 290.35 | 290.35 |
| NaCl, Powder | 150 | 150 | 150 |
| Croscarmellose Sodium | 150 | — | — |
| Crospovidone | — | 150 | — |
| Sodium Starch Glycolate | — | — | 150 |
| Colloidal Silicon Dioxide | 3.75 | 3.75 | 3.75 |
| Magnesium Stearate (Non-Bovine) | 7.5 | 7.5 | 7.5 |
| Total | 1485 | 1485 | 1485 |
| Extra-Granular | | | |
| Magnesium Stearate (Non-Bovine) | 15 | 15 | 15 |
| Total | 1500 | 1500 | 1500 |

TABLE 10
Mechanical Properties and Disintegration Times for Tablets
Containing Two Co-Granulated SDIs, a Crystalline API
and Disintegration System Using Varying Disintegrants

| | | | Disintegration |
|---|---|---|---|
| Disintegrant | Pressure (MPa) | Hardness (kP) | Time (mm:ss) |
| Croscarmellose | 100 | 28.8 | 7:21 |
| Sodium | 130 | 36.4 | 17:06 |
| | 180 | 38.9 | 14:14 |
| Crospovidone | 100 | 30 | 15:12 |
| | 140 | 37.1 | 25:55 |
| | 190 | 41.7 | 34:04 |
| Sodium Starch | 105 | 24.3 | 27:22 |
| Glycolate | 145 | 31.2 | 34:57 |
| | 250 | 36.5 | >40:00 |

### Example 8

Compound I, copovidone, and vitamin E TPGS was dissolved in acetone at a solids loading of 3.5% percent. The solids were comprised of 30% Compound I, 60% copovidone and 10% vitamin E TPGS. The resulting solution was spray dried on a PROCEPT spray dryer and secondary dried in a vacuum oven. The spray dried dispersion was blended with the intragranular components shown in Table 11 on a laboratory mixer (TURBULA®) for 10 min. at 46 rpm followed by lubrication with magnesium stearate for 5 min. at 46 rpm. Samples with various levels of disintegrant and salt were prepared. The resultant blends were slugged on an

MTS compression machine using ¾ inch knurled tooling to produce slugs of approximately 2 mm in thickness and a tensile strength of 1.0 MPa. Granules were formed by pushing the slugs through a 1 mm mesh screen and blended with extragranular excipients and lubricated with extragranular magnesium stearate for 10 min. and 5 min., respectively, on a laboratory mixer (TURBULA®) at 46 rpm. Tablets of 300 mg were compressed to tensile strengths of 1.5 MPa to 2.0 MPa on a compaction study machine (by Roland Research Devices Incorporated) using 12/32″ round standard concave tooling. The disintegration time of the resulting tablets was measured using a standard USP reciprocating disintegration apparatus with cylinders in 900 mL of SGF at 37° C. Table 12 illustrates the decrease in disintegration time with the addition of NaCl to the formulation over just increasing levels of croscarmellose sodium (CCNa). As shown in Table 13, this allowed the dispersion load to be increased from 33% to at least 50% in the tablet while maintaining disintegration times at approximately 25 min. and enabling the development of fixed-dose combination tablets.

TABLE 11
Compositions of Tablets Containing Compound I

| Formulation Composition | % w/w | % w/w |
|---|---|---|
| SDI: 30% Compound I, 10% vitamin E TPGS, | 33.3 | 33.3 |
| 60% copovidone | | |
| Microcrystalline cellulose | 31.33 | 21.83 |
| Lactose, Monohydrate | 31.33 | 21.83 |
| Croscarmellose sodium | 3.0 | |
| Croscarmellose sodium (intragranular) | | 6.0 |
| NaCl (intragranular) | | 10.0 |
| Magnesium stearate (intragranular) | 0.25 | 0.25 |
| Colloidal silicon dioxide (intragranular) | .5 | .5 |
| Croscarmellose sodium (extragranular) | | 6.0 |
| Magnesium stearate (extragranular) | 0.25 | 0.25 |
| Total | 100 | 100 |

TABLE 12
Disintegration Time for Tablets Containing Compound I

| | Disintegration |
|---|---|
| Formulation | Time in SGF (min) |
| Base formulation | 19 |
| Base + 3% extragranular croscarmellose sodium | 25 |
| Base + 7% extragranular croscarmellose sodium | 20 |
| Base + 12% extragranular croscarmellose sodium | 11 |
| Base + 8% NaCl + 7% extragranular croscarmellose | 7 |
| sodium | |

TABLE 13
SDI Loading in Tablets Containing Compound I

| Formulation Composition | % w/w |
|---|---|
| SDI: 30% Compound I, 10% vitamin E TPGS, 60% copovidone | 50.00 |
| Microcrystalline cellulose | 13.50 |
| Lactose, Monohydrate | 13.50 |
| Croscarmellose sodium (intragranular) | 6.0 |
| NaCl (intragranular) | 10.0 |
| Magnesium stearate (intragranular) | 0.25 |
| Colloidal silicon dioxide (intragranular) | 0.5 |
| Croscarmellose sodium (extragranular) | 6.0 |
| Magnesium stearate (extragranular) | 0.25 |
| Total | 100 |
| Disintegration time in SGF | 25 min |

### Example 9

In one formulation, Compound L was blended with copovidone at a 30:70 ratio in a V-shell dry blender (PATTERSON-KELLY®) at 25 rpm for 10 min. and extruded on a twin screw extruder (THERMO-ELECTRON PHARMALAB 16®) at a product temperature of approximately 150° C. The resulting extrudate was milled in a blade mill (FITZMILL®, model L1A) at 3000 rpm with impact blades and a 500 micron screen to produce an extrudate with a mean particle size of approximately 200 micron. The milled extrudate was blended with excipients in a laboratory mixer (Turbula®) at 46 rpm for 10 min. followed by lubrication with sodium stearyl fumarate at 46 rpm for 5 min. The blend was compressed into tablets at 250 mg image weight using 9/32″ or 10/32″ standard round concave tooling on a universal material testing machine (MTS INSTRON®) at 150 MPa compaction pressure. The disintegration time of the resulting tablets was measured using a standard USP reciprocating disintegration apparatus with cylinders in 900 mL of low pH aqueous medium (1 L water, 10 mL concentrated 0.01 N HCl) at 37° C. Table 14 shows a disintegration time of 21:56 mm:ss without salt, while Table 15 shows a disintegration time of 3:46 mm:ss with salt in a similar composition. This allows for an increase in extrudate load to >50% and a smaller image to be made at a given dose without hindering drug release.

TABLE 14
Compositions of Tablets of Compound L and
Disintegration Times with High Disintegrant Levels

| | Composition | |
|---|---|---|
| Material Description | (%) | mg/unit |
| INT Compound L 300 mg/g, milled (HME) | 53.33 | 133.3 |
| Cellulose, Microcrystalline, Compendial | 14.67 | 36.67 |
| Mannitol, Compendial | 22.00 | 55.00 |
| Croscarmellose Sodium, Compendial | 9.000 | 22.50 |
| Sodium Stearyl Fumarate, Compendial | 1.000 | 2.50 |
| Total | 100.0 | 250.0 |

| Tablet | Time (m:s) |
|---|---|
| 1 | 19:13 |
| 2 | 22:53 |
| 3 | 23:43 |
| Mean | 21:56 |

TABLE 15
Compositions of Tablets of Compound L and
Disintegration Times with Disintegrant and Salt

| | Composition | |
|---|---|---|
| Material Description | (%) | mg/unit |
| INT Compound L 300 mg/g, milled (HME) | 53.33 | 133.3 |
| Cellulose, Microcrystalline, Compendial | 10.67 | 26.67 |
| Mannitol, Compendial | 16.00 | 40.00 |
| Sodium Chloride, Compendial | 10.00 | 25.00 |
| Croscarmellose Sodium, Compendial | 9.000 | 22.50 |
| Sodium Stearyl Fumarate, Compendial | 1.000 | 2.50 |
| Total | 100.0 | 250.0 |

| Tablet | Time (m:s) |
|---|---|
| 1 | 3:54 |
| 2 | 3:37 |
| Mean | 3:46 |

In another formulation, Compound L was blended with copovidone and vitamin E TPGS at a 30:5:65 ratio in a V-shell dry blender (PATTERSON-KELLY®) at 25 rpm for 10 min. and extruded on a twin screw extruder (THERMO-ELECTRON PHARMALAB 16®) at a product temperature of approximately 150° C. The resulting extrudate was milled in a blade mill (FITZMILL®, model L1A) at 3000 rpm with impact blades and a 500 micron screen to produce an extrudate with a mean particle size of approximately 200 micron. The milled extrudate was blended with excipients in a laboratory mixer (TURBULA®) at 46 rpm for 10 min. followed by lubrication with sodium stearyl fumarate at 46 rpm for 5 min. The blend was compressed into tablets at 250 mg image weight using 9/32″ or 10/32″ standard round concave tooling on a compaction study machine (by Roland Research Devices Incorporated) or a universal material testing machine (MTS IINSTRON®) at 175 MPa compaction pressure. The disintegration time of the resulting tablets was measured using a standard USP reciprocating disintegration apparatus with cylinders in 900 mL of low pH aqueous medium (1 L water, 10 mL concentrated 0.01 N HCl) at 37° C. Table 16 shows a disintegration time of 43:34 mm:ss without salt, while Table 17 shows a disintegration time of 5:58 mm:ss with salt in a similar composition. This allows for an increase in surfactant-containing extrudate load to >50% and a smaller image to be made at a given dose without hindering drug release.

TABLE 16
Compositions of Tablets of Compound L and
Disintegration Times with High Disintegrant Levels

| | Composition | |
|---|---|---|
| Material Description | (%) | mg/unit |
| INT Compound L 300 mg/g, milled (HME, | 53.33 | 133.3 |
| Vitamin E TPGS) | | |
| Cellulose, Microcrystalline, Compendial | 25.78 | 64.44 |
| Mannitol, Compendial | 12.89 | 32.22 |
| Croscarmellose Sodium, Compendial | 6.000 | 15.00 |
| Sodium Stearyl Fumarate, Compendial | 2.000 | 5.000 |
| Total | 100.0 | 250.0 |

| | Time |
|---|---|
| Tablet | (mm:ss) |
| 1 | 43:57 |
| 2 | 42:11 |
| 3 | 44:35 |
| Mean | 43:34 |

TABLE 17
Compositions of Tablets of Compound L and
Disintegration Times with Disintegrant and Salt

| | Composition | |
|---|---|---|
| Material Description | (%) | mg/unit |
| INT Compound L 300 mg/g, milled (HME, | 53.33 | 133.3 |
| Vitamin E TPGS) | | |
| Cellulose, Microcrystalline, Compendial | 10.67 | 26.67 |
| Mannitol, Compendial | 16.00 | 40.00 |
| Sodium Chloride, Compendial | 10.00 | 25.00 |
| Croscarmellose Sodium, Compendial | 9.000 | 22.50 |
| Sodium Stearyl Fumarate, Compendial | 1.000 | 2.500 |
| Total | 100.0 | 250.0 |

| | Time |
|---|---|
| Tablet | (mm:ss) |
| 1 | 6:18 |
| 2 | 5:38 |
| Mean | 5:58 |

### Example 10

Compound J was extruded at a 20% drug load with 5% vitamin E TPGS and 75% copovidone on a 16 mm thermo electron extruder at a product temperature of approximately 160° C. A series of experiments studied disintegrant types and combinations (croscarmellose sodium, crospovidone, calcium silicate) with and without the addition of salt (NaCl) at a 50% dispersion load. Tablets were compressed on the MTS compression machine to tensile strengths of 1.75 MPa to 3.0 MPa and compared by dissolution. Tablets containing salt show a dramatic improvement in dissolution in SGF over tablets containing a disintegrant alone. After 15 min., 92% of the Compound J was released in SGF when salt was added to a formulation containing croscarmellose sodium versus 61% without salt. Likewise, 94% of Compound J was released at 15 min. in SGF when salt was added to a formulation containing crospovidone versus 42% without salt.

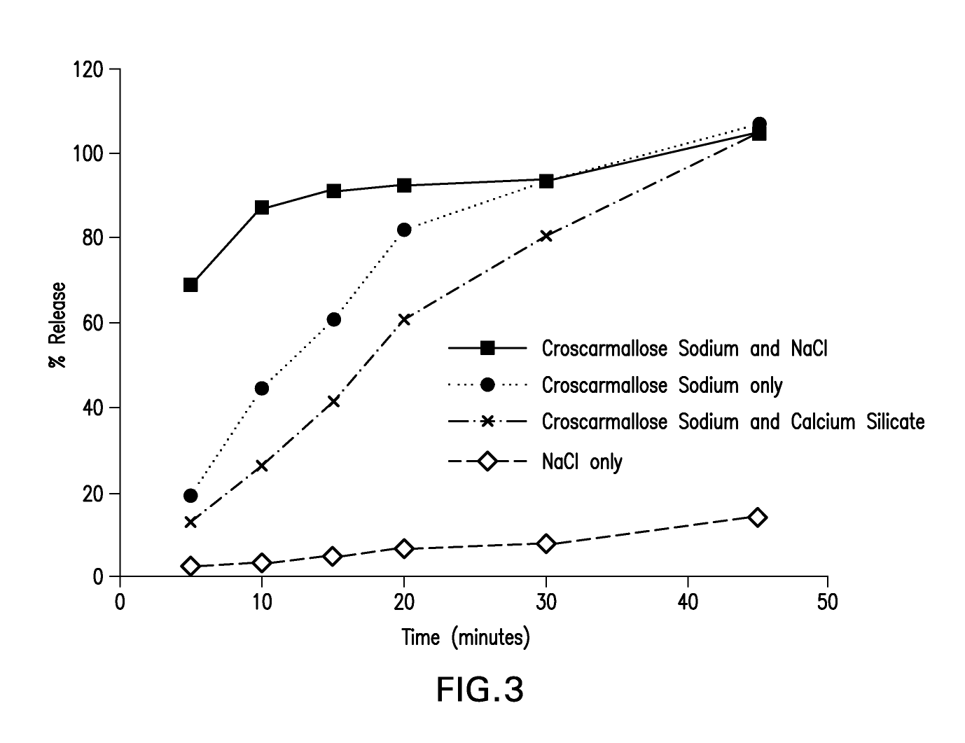

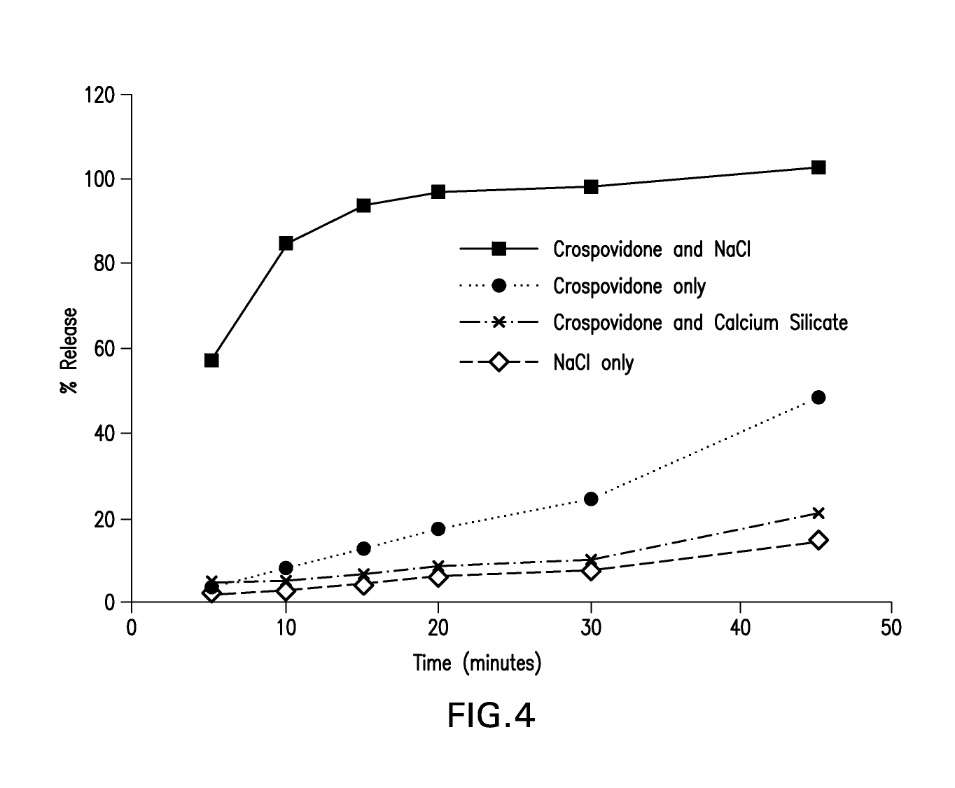

FIGS. 3 and 4 show the comparative release rates of 50 mg of Compound J from formulations containing combinations of disintegrant (croscarmellose sodium or crospovidone) and NaCl. Dissolution conditions are USP 2 paddles, 50 rpm, 37° C., in 900 mL SGF. The surprising synergstic effects of the disintegrant and particulate salt combination can be seen from these figures.

### Example 11

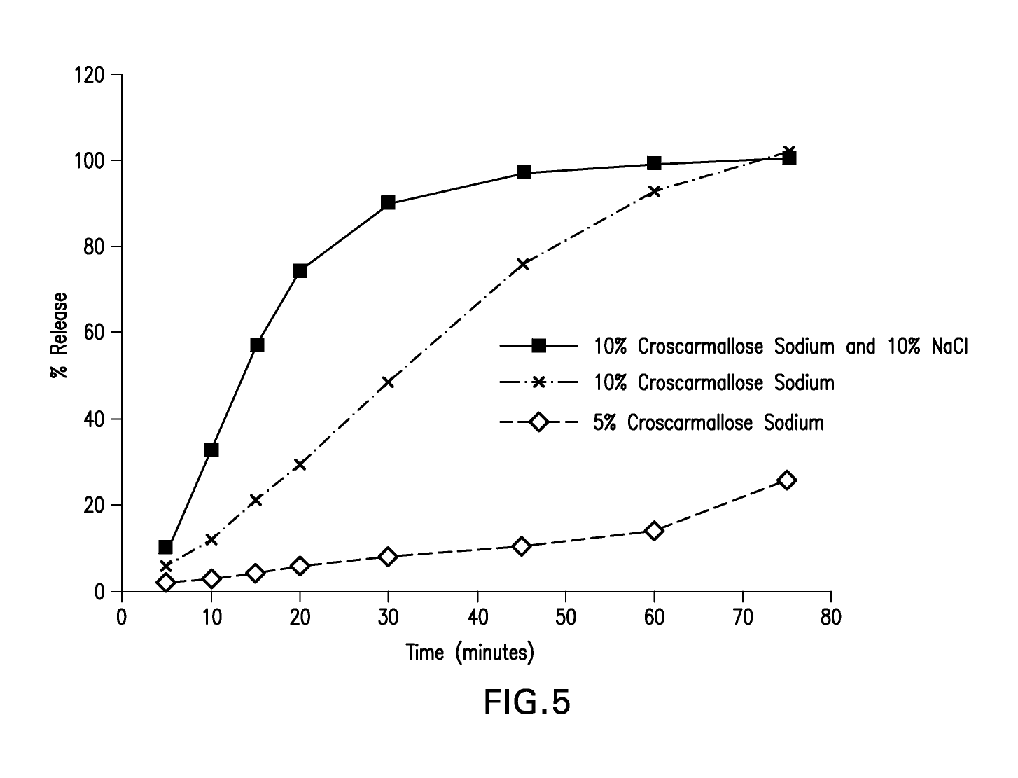

Compound K was extruded at a 23% drug load with 5% vitamin E TPGS and 75% copovidone on a 27 mm LIESTRITZ extruder at a product temperature of approximately ˜195° C. Formulations with 5% and 10% CCNa with and without NaCL were compressed on a compaction study machine (by Roland Research Devices Incorporated) and tested for Compound K release by dissolution in SGF. FIG. 5 shows that NaCl increases the dissolution rate of Compound K over tablets containing only disintegrant.

FIG. 5 shows the comparative dissolution profiles of 52 mg of Compound K from formulations containing combinations of disintegrant and NaCl. Dissolution conditions are USP 2 paddles, 50 rpm, 37° C., in 900 mL SGF. The surprising synergstic effects of the disintegrant and particulate salt combination can be seen from FIG. 5.

### Example 12

Compound J formulations shown in Table 18 were blended and lubed for compression on an RRDI compaction simulator. Hardness and volume of the tablets were controlled by varying compaction pressure between 100 MPa to 200 MPa to eliminate any tablet property variation/effects, as shown in Table 19. The disintegration time of the resulting tablets was measured using a standard USP reciprocating disintegration apparatus with cylinders in 900 mL of SGF at 37° C. Among all excipients, powder NaCl was unexpectedly found to be the most efficient in accelerating disintegration based on solubility and ionic strength considerations (Table 20). The inorganic salts likely prevent gel formation through inhibiting quick release of copovidone in the aqueous media to form a high concentration gel layer. Though some ions can shorten the tablet disintegration more efficiently than chloride ion (Cl⁻), rapidly reaching high local concentration is critical.

TABLE 18
Formulations of Compound J

| | F1, % | F2, % | F3, % | F4, % | F5, % | F6, % | F7, % | F8, % | F9, % | F10, % |
|---|---|---|---|---|---|---|---|---|---|---|
| HME/copovidone | 45 | 45.5 | 45.5 | 45.5 | 45.5 | 45.5 | 45.5 | 45.5 | 45.5 | 45.5 |
| Microcrystalline | 49.75 | 40.7 | 40.7 | 40.7 | 40.7 | 40.7 | 40.7 | 40.7 | 40.7 | 40.7 |
| cellulose | | | | | | | | | | |
| CCNa | 5 | 4.5 | 4.5 | 4.5 | 4.5 | 4.5 | 4.5 | 4.5 | 4.5 | 4.5 |
| Magnesium | 0.25 | 0.2 | 0.2 | 0.2 | 0.2 | 0.2 | 0.2 | 0.2 | 0.2 | 0.2 |
| stearate | | | | | | | | | | |
| NaCl | | 9.1 | | | | | | | | |
| NaHCO₃ | | | 9.1 | | | | | | | |
| Na₂CO₃ | | | | 9.1 | | | | | | |
| Na₂SO₄ | | | | | 9.1 | | | | | |
| Na₂HPO₄ | | | | | | 9.1 | | | | |
| NaCl, powder | | | | | | | 9.1 | | | |
| Lactose | | | | | | | | 9.1 | | |
| Sucrose | | | | | | | | | 9.1 | |
| K₂CO₃ | | | | | | | | | | 9.1 |

TABLE 19
Disintegration times for Formulations of Compound J

| | Hardness, kP | Thickness, mm |
|---|---|---|
| Control, 45% HME, 100 MPa | 8.7 | 3.13 |
| Control, 45% HME, 150 MPa | 10.9 | 2.98 |
| Powder NaCl, 45% HME, 150 MPa | 8.2 | 2.92 |
| K₂CO₃, 45% HME, 150 MPa | 8.7 | 2.98 |
| NaCl, 45% HME, 200 MPa | 8.8 | 2.94 |
| NaHCO₃, 45% HME, 150 MPa | 8.6 | 2.96 |
| Na₂CO₃, 45% HME, 150 MPa | 7.9 | 2.93 |
| Lactose, 45% HME, 125 MPa | 8.7 | 3.00 |
| Sucrose, 45% HME, 125 MPa | 8.6 | 3.00 |
| Na₂SO₄, 45% HME, 150 MPa | 8.4 | 2.89 |
| Na₂HPO₄, 45% HME, 150 MPa | 8.9 | 2.96 |
| Control, 50% HME, crospovidone | 8.9 | 3.00 |

TABLE 20
Solubility and Ionic Strength of Formulations of Compound J

| | | 40° C. | Ionic |
|---|---|---|---|
| | SGF, min | Solubility (g/L) | Strength (M) |
| Control, 45% HME | | | |
| Control, 45% HME | >1 h | | |
| Powder NaCl, 45% HME | 1.5 | 359 | 0.015 |
| K₂CO₃, 45% HME, | 6 | 1170 | 0.019 |
| NaCl, 45% HME | 18 | 359 | 0.015 |
| NaHCO₃, 45% HME | 17 | 127 | 0.011 |
| Na₂CO₃, 45% HME | 13 | 490 | 0.025 |
| Lactose, 45% HME | >1 h | 216 | |
| Sucrose, 45% HME | >1 h | 2000 | |
| Na₂SO₄, 45% HME | 20 | 490 | 0.019 |
| Na₂HPO₄, 45% HME | 22 | 93 (20° C.) | 0.015 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A pharmaceutical oral dosage form, comprising:
a) one or more solid dispersion formulations, each independently comprising
i) one or more active pharmaceutical ingredients,
ii) one or more pharmaceutically acceptable polymers, and
iii) optionally one or more pharmaceutically acceptable surfactants, and
wherein said one or more active pharmaceutical ingredients and said one or more optional surfactants are dispersed in a polymer matrix formed by said one or more pharmaceutically acceptable polymers; and
b) a disintegrant selected from the group consisting of modified starches, cross-linked polyvinylpyrrolidones, modified celluloses, soy polysaccharides, cross-linked alginic acids, gellan gum, xantham gum, calcium silicate and ion exchange resins; and
c) an inorganic salt, where the inorganic salt is in the form of particles, wherein said particles are characterized by (i) a d₅₀ value of less than about 325 micron; (ii) a d₁₀ value of less than about 185 micron; and (iii) a d₉₀ value of less than about 470 micron;
d) optionally one or more additional active pharmaceutical ingredients; and
e) optionally one or more excipients selected from the group consisting of diluents, additional disintegrants, additional salts, lubricants, glidants, sweetening agents, flavoring agents, coloring agents, preserving agents, binding agents, and antioxidants;
wherein the disintegrant and the inorganic salt are provided in a ratio of from about 2:1 to about 1:3; and
wherein said pharmaceutical oral dosage form is a tablet or a capsule.

2. The pharmaceutical oral dosage form according to claim 1, wherein the one or more solid dispersion formulations, disintegrant and inorganic salt are in the form of a granulation intermediate.

3. The pharmaceutical oral dosage form according to claim 1, wherein the one or more solid dispersion formulations, disintegrant and inorganic salt are in the form of a blended composition.

4. The pharmaceutical oral dosage form according to claim 1, wherein the disintegrant is selected from the group consisting of sodium carboxylmethyl starch, crospovidone, croscarmellose sodium, calcium silicate and ion exchange resins.

5. The pharmaceutical oral dosage form according to claim 4, wherein the disintegrant is selected from the group consisting of crospovidone and croscarmellose sodium.

6. The pharmaceutical oral dosage form according to any one of claims 1 to 5, wherein the inorganic salt is selected from the group consisting of sodium chloride, potassium chloride, potassium carbonate, sodium carbonate, sodium bicarbonate, sodium sulfate and sodium phosphate (dibasic).

7. The pharmaceutical oral dosage form according to claim 1, wherein the inorganic salt is selected from the group consisting of sodium chloride and potassium chloride.

8. The pharmaceutical oral dosage form according to claim 1, wherein the inorganic salt is in the form of powder, wherein said powder is characterized by (i) a d₅₀ value of less than about 210 micron; (ii) a d₁₀ value of less than about 50 micron; and (iii) a d₉₀ value of less than about 470 micron.

9. The pharmaceutical oral dosage form according to claim 1, wherein the disintegrant and the inorganic salt are provided in a 1:1 ratio.

10. The pharmaceutical oral dosage form according to claim 1, wherein the one or more pharmaceutical active agent is selected from the group consisting of poorly soluble drugs.

11. The pharmaceutical oral dosage form according to claim 10, wherein the one or more pharmaceutical active agent is selected from the group consisting of HCV NS3/NS4a inhibitors, HCV NS5a inhibitors, HCV NS5b inhibitors, HIV inhibitors, calcitonin gene-related peptide antagonist compounds, and orexin receptor antagonists.

12. The pharmaceutical oral dosage form according to claim 11, wherein the one or more pharmaceutical active agents are one or more agents independently selected from the group consisting of
(1aR,5S,8S,10R,22aR)-N-[(1R,2S)-1-[(cyclopropylsulfonamido)carbonyl]-2-ethenylcyclopropyl]-14-methoxy-5-(2-methylpropan-2-yl)-3,6-dioxo-1,1a,3,4,5,6,9,10,18,19,20,21,22,22a-tetradecahydro-8H -7,10-methanocyclopropa[18,19][1,10,3,6]dioxadiazacyclononadecino[11,12-b]quinoxaline-8-carboxamide hydrate: 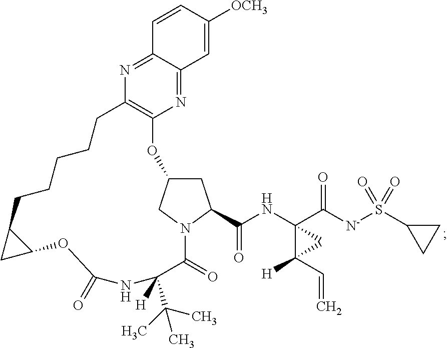
(5R,7S,10S)-10-tert-Butyl-N-{(1R,2R)-1-[N -(cyclopropanesulfonyl)carbamoyl]-2-ethylcyclopropyl}-15,15-dimethyl-3,9,12-trioxo -6,7,9,10,11,12,14,15,16,17,18,19-dodecahydro-1H,3H,5H -2,23 :5,8-dimethano -4,13,2,8,11-benzodioxatriazacyclohenicosine-7-carboxamide: 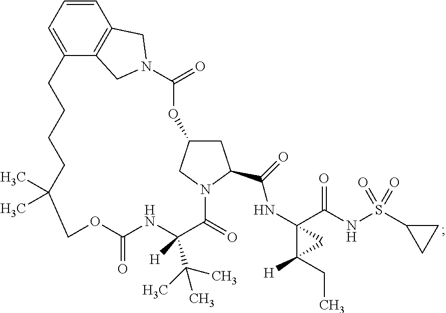
(1R,5S)-N-[3-Amino-1-(cyclobutylmethyl)-2,3 -dioxopropyl]-3 -[2(S) -[[[(1,1-dimethylethyl)amino]carbonyl]amino]-3,3-dimethyl-1-oxobutyl]-6, 6-dimethyl-3-azabicyclo[3.1.0]hexan-2(S)-carboxamide: 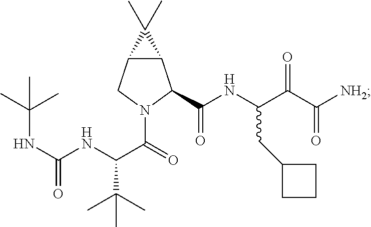 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]
(1R,2 S,5 S)-3-((S)-2-(3-(1-((tert-butyl sulfonyl)methyl)cyclohexyl)ureido) -3,3-dimethylbutanoyl)-N-((S)-1-(cyclopropylamino)-1,2-dioxoheptan-3 -yl)-6,6-dimethyl -3-azabicyclo[3.1.0]hexane-2-carboxamide: 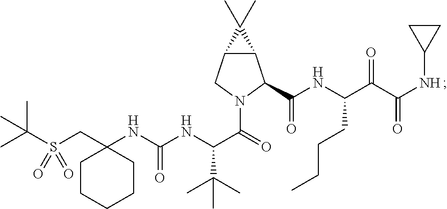 [SMILES: CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]
dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate: 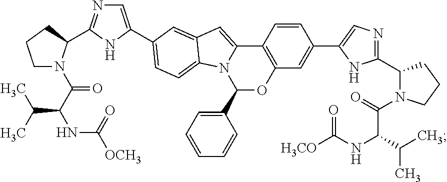 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]
dimethyl ((2S,2′S)-((2S,2′S)-2,2′-(5,5′-((S)-6-(2-cyclopropylthiazol-5-yl) -1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate: 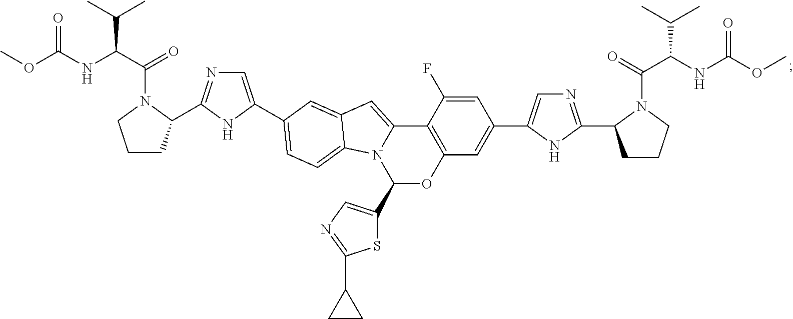
(2R)-isopropyl 2-(((((2R,3R,4R,5R)-4-chloro-5-(2,4-dioxo-3 ,4-dihydropyrimidin-1(2H)-yl)-3-hydroxy-4-methyltetrahydrofuran-2-yl)methoxy)(phenoxy)phosphoryl)amino)propanoate: 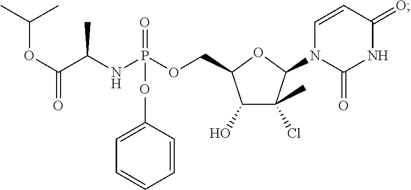 [SMILES: CC(C)OC(=O)[C@@H](C)NP(=O)(OC[C@H]1O[C@@H](n2ccc(=O)[nH]c2=O)[C@](C)(Cl)[C@@H]1O)Oc1ccccc1]
(2R,3S,4R,5R)-4-cyano-5-(2,4-dioxo-3,4-dihydropyrimidin-1(2H)-yl)-2-((((((S)-1isopropoxy-1-oxopropan-2-yl)amino)(phenoxy)phosphoryl)oxy)methyl)-4-methyltetrahydrofuran-3-yl isobutyrate: 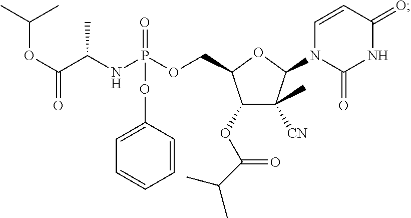 [SMILES: CC(C)OC(=O)[C@H](C)NP(=O)(OC[C@H]1O[C@@H](n2ccc(=O)[nH]c2=O)[C@](C)(C#N)[C@@H]1OC(=O)C(C)C)Oc1ccccc1]
5-(11-fluoro-6H-pyrido[2′,3′:5,6][1,3]oxazino[3,4-a]indol-2-yl)-2-(4-fluorophenyl)-N-methyl-6-(N-methylmethylsulfonamido)benzofuran-3-carboxamide: 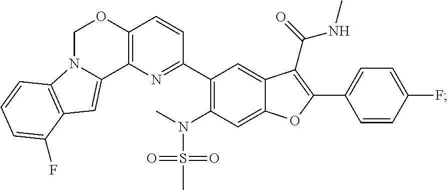 [SMILES: CNC(=O)c1c(-c2ccc(F)cc2)oc2cc(N(C)S(C)(=O)=O)c(-c3ccc4c(n3)-c3cc5c(F)cccc5n3CO4)cc12]
(S)-N-((3S,5S,6R)-6-methyl-2-oxo-5-phenyl-1-(2,2,2-trifluoroethyl)piperidin-3-yl)-2′-oxo-1′,2′,5,7-tetrahydrospiro[cyclopenta[b]pyridine-6,3′-pyrrolo[2,3-b]pyridine]-3-carboxamide: 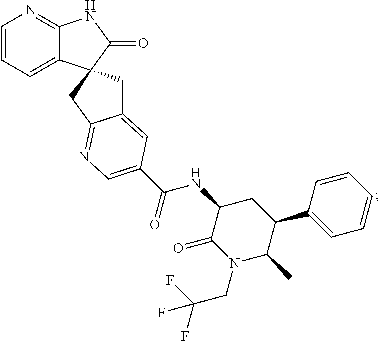 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]
(S)-N-((3S,5S,6R)-6-methyl-2-oxo-1-(2,2,2-trifluoroethyl)-5-(2,3 ,6-trifluorophenyl)piperidin-3-yl)-2′-oxo-1′,2′,5,7-tetrahydrospiro[cyclopenta[b]pyridine -6,3′-pyrrolo[2,3-b]pyridine]-3 -carboxamide): 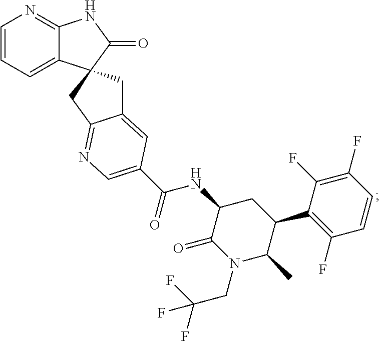 [SMILES: C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]
(5-chloro-2-{(5R)-5-methyl-4-[5-methyl-2-(2H-1,2,3-triazol2-yl)benzoyl]-1,4-diazepan-1-yl}-1,3-benzoxazole: 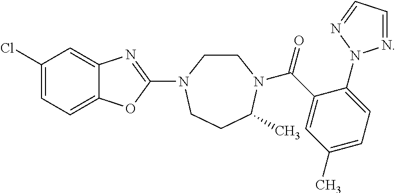 [SMILES: Cc1ccc(-n2nccn2)c(C(=O)N2CCN(c3nc4cc(Cl)ccc4o3)CC[C@H]2C)c1]
and
2-{2-[((2R,5R)-5-{[(5-fluoropyridin-2-yl)oxy]methyl}-2-methylpiperidin -1-yl)carbonyl]-4-methylphenyl}pyrimidine:

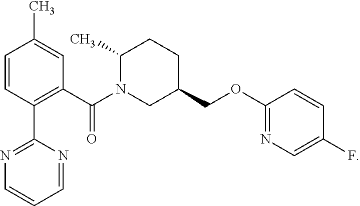 [SMILES: Cc1ccc(-c2ncccn2)c(C(=O)N2C[C@H](COc3ccc(F)cn3)CC[C@H]2C)c1]

13. The pharmaceutical oral dosage form according to claim 1, wherein the one or more pharmaceutically acceptable polymers are one or more independently selected from the group consisting of copovidone, HPMC and combinations thereof and the one or more solid dispersion formulations are stabilized amorphous dispersion.

14. The pharmaceutical oral dosage form according to claim 1, wherein the solid pharmaceutical dosage form is a tablet, and the tablet is film-coated.

15. The pharmaceutical dosage form according to claim 1, wherein the disintegrant comprises from about 3% w/w to about 15% w/w of solid pharmaceutical dosage form and the salt comprises from about 3% w/w to about 15% w/w of solid pharmaceutical dosage form.

16. The pharmaceutical dosage form according to claim 1, wherein the disintegrant comprises from about 5% w/w to about 10% w/w of solid pharmaceutical dosage form and the salt comprises from about 5% w/w to about 10% w/w of solid pharmaceutical dosage form.
